(19) United States
(12) Patent Application Publication
(10) Pub. No.: US 2025/0031563 A1
(43) Pub. Date: Jan. 23, 2025

(54) Determining dynamics of excitations in materials

(71) Applicant: Scientific Computing & Modelling Holding B.V., Amsterdam (NL)

(72) Inventors: Mahyar Taherpour, Eindhoven (NL); Clint van Hoesel, Eindhoven (NL); Reinder Coehoorn, Eindhoven (NL); Peter Arnold Bobbert, Nuenen (NL)

(73) Assignee: Scientific Computing & Modelling Holding B.V., Amsterdam (NL)

(21) Appl. No.: 18/711,112

(22) PCT Filed: Nov. 17, 2022
(86) PCT No.: PCT/NL2022/050658

§ 371 Date: May 17, 2024

(30) Foreign Application Priority Data
Nov. 18, 2021 (EP) 21209106.0

(51) Int. Cl. H10K 71/70; G06F 30/367; G06F 111/08; G16C 60/00; H01L 21/66

(52) U.S. Cl. CPC H10K 71/70; G06F 30/367; G16C 60/00; H01L 22/34; G06F 2111/08

(57) ABSTRACT

A method of determining a correlated evolution of a state relating to a solid object, the method involving obtaining information indicative of a plurality of properties of the solid object. The solid object includes at least one material, and the plurality of properties include material properties of the at least one material. Information is obtained indicative of a certain excitation of the solid object. A closed set of hierarchical equations representing dynamics of the interacting excitations within the solid object, including spatial correlations among the plurality of excitations, based on the properties of the solid object are determined; the hierarchical equations are indicative of a time evolution of correlation functions of the excitations, and the set of closed hierarchical equations is closed according to an approximation. The correlated evolution of the state of the solid object based on the closed set of hierarchical equations and the certain excitation is also determined.

### FIELD OF THE INVENTION

The invention relates to a method and system for determining a correlated evolution of a state relating to a solid object. The invention further relates to a computer program.

### BACKGROUND OF THE INVENTION

Simulations of the properties and behavior of materials within electronic devices such as light emitting diodes, light sensors, transistors and batteries are nowadays employed in industry to speed up the research and development of new device technologies, the manufacturing of more efficient devices and the synthesis of better performing materials. A simulation assisted workflow can support the engineers in creating devices and materials with better stability, lifetime and performance. In this way, expensive trial-and-error experimentation is minimized, thus leading to a shorter time-to-market.

To understand and predict the behavior of ever more complex materials and devices, it is desirable to simulate the fundamental physical and chemical processes down to the nanoscale and with atomistic level accuracy. These simulations are very demanding, but necessary to provide the simulation with prediction capability so to bring effective added value for the user of the simulation results.

A difficult challenge is to correctly determine the macroscopic response (emergent property) of materials that undergo a physical external stimulus (e.g. under light illumination, or under the application of a voltage bias). This is even more difficult when materials are mixed and stacked in many layers to create a complex electronic device configuration. The prediction of the device physical behavior given the device structure and the fundamental properties of the materials composing the device can be done by computing the dynamics of the excitations that happen at the nanoscale within the material and simulating their impact on the device physical behavior. The accurate determination of such excitation dynamics can only be achieved when properly accounting for correlations among these excitations. Failure to do that would lead to approximate or erroneous predictions about the properties and functionalities of the systems under study.

Simulations of the dynamics of excitations are currently done using modelling techniques that are accurate but computationally demanding, like kinetic Monte Carlo (KMC), or using less computationally demanding faster methods that ignore correlation effects, like Master Equation (ME) modelling using the mean field approximation (MFA) or one-dimensional drift-diffusion (1D-DD) modelling. KMC can correctly describe correlation effects because only one event is executed at a time. For the same reason, KMC computations are known to be hard to parallelize, which makes them very slow. This is why different methods that are better parallelizable and fast are of great interest.

The present invention seeks to address the limitations of using faster simulation methods while correctly and efficiently determining the correlations in the excitations of the system.

The time-evolution of the spatial correlation among the charges within a material (i.e. charged excitations, like electrons, holes and ions) can be described using a hierarchical set of coupled equations with increasing order, so-called Boguliobov-Born-Green-Kirkwood-Yvon (BBGKY) equations, as demonstrated by A. M. Shumilin and Y. M. Beltukov in 2019 (A. M. Shumilin and Y. M. Beltukov, “System of correlation kinetic equations and the generalized equivalent circuit for hopping transport”, Physical Review B 100, 014202 (2019)). This method, however, is shown to quickly become intractable for practical purposes so that achieving the desired accuracy becomes less competitive with KMC methods.

J. G. Kirkwood in 1935 discloses a superposition approximation (SA) method in the context of a theoretical study of spatial correlation functions in liquids (J. G. Kirkwood, “Statistical mechanics of fluid mixtures”, The Journal of Chemical Physics 3, 300 (1935)). The name “superposition approximation” for this approximation was introduced by J. G. Kirkwood and E. M. Boggs in 1942 (J. G. Kirkwood and E. M. Boggs, “The radial distribution function in liquids”, The Journal of Chemical Physics 10, 394 (1942)). However, this approximation has never been developed to be applicable in the context of solid materials, such as (opto) electronic devices or batteries.

WO 2020/021008 A1 discloses three-dimensional master equation modeling for disordered semiconductor devices. Charge transport is modeled as incoherent hopping between localized molecular states, and recombination is modeled as a nearest-neighbor process where an electron at a first location and a hole at a second location can recombine at either the first location or the second location. Here the first and second locations are any pair of nearest neighbor locations. The recombination rate is modeled as a product of a prefactor Y, hopping rates and state occupancies.

“Three-Dimensional Modeling of Bipolar Charge-Carrier Transport and Recombination in Disordered Organic Semiconductor Devices at Low Voltages”, by Feilong Liu, Harm van Eersel, Peter A. Bobbert, and Reinder Coehoorn, in Phys. Rev. Applied 10, 054007, 2018 describes a systematic method for extending 3D ME simulations to bipolar devices. The electroluminescence from organic light-emitting diodes can be predicted with molecular-scale resolution using three-dimensional kinetic Monte Carlo (3D KMC) simulations. However, around and below the built-in voltage KMC simulations are computationally inefficient. 3D master-equation (3D ME) simulation methods, which are fastest for low voltages, are so far mainly available for describing unipolar charge transport. In such simulations, the charge-carrier interactions are treated within a mean-field approach.

### SUMMARY OF THE INVENTION

It is an object of the present invention to provide an improved determination of the correlations among the excitations in a solid object.

According to a first aspect of the invention, a method is provided for determining a correlated evolution of a state relating to a solid object subjected to a stimulus, the method comprising
obtaining information indicative of a composition and geometry of the solid object;
obtaining information indicative of an external stimulus in respect of the solid object;
determining a set of hierarchical equations representing dynamics of excitations within the solid object, including spatial correlations among the plurality of excitations, based on the composition and geometry of the solid object, wherein the hierarchical equations are indicative of a time evolution of correlation functions of the excitations,
determining a closed set of hierarchical equations by closing the set of hierarchical equations according to an approximation; and
determining the correlated evolution of the state of the solid object based on the closed set of hierarchical equations and the external stimulus.

The closed set of hierarchical equations allows a relatively efficient computation by not having to consider higher order terms. Moreover, by virtue of the estimation, the evolution may be determined with relatively high accuracy.

The solid object may be an organic semiconductor device. The method can advantageously efficiently provide highly accurate and relevant information regarding the evolution of the state of the object, in respect of organic semiconductor devices.

The solid object may be a part of an electronic device. For example, the solid object may be a component of an electronic device. This feature allows the method to provide purposeful information about the operation and/or performance of the electronic device.

The method may comprise predicting a physical behavior of the solid object based on the correlated evolution of the state of the solid object, wherein the physical behavior is associated with at least one of: light emitted by the solid object, light absorbed by the solid object, energy stored within the solid object, energy absorbed by the solid object and energy released by the solid object. This feature allows to purposefully control the solid object by determining what external excitation can achieve a desired physical effect. Further, the feature allows to design the solid object in view of its physical behavior.

The method may comprise performing a measurement in respect of the solid object to generate at least part of the information. By performing a measurement, the current state of the solid object as measured can be taken into account, so that the determined correlated evolution corresponds to what is actually happening in the solid object.

The method may comprise manufacturing the solid object in dependence on the determined correlated evolution of the state of the solid object. This allows to test the performance of several imaginary designed solid objects before actually manufacturing a selected solid object that has the desired properties according to the determined evolution.

The method may further comprise repeating the steps of determining the set of closed hierarchical equations and determining the correlated evolution of the state of the solid object in respect of a plurality of different combinations of properties, and selecting the solid object to be manufactured based on the determined correlated evolution of the state of the solid object. The selection may involve comparing the determined correlated evolution to a predetermined desired correlated evolution.

The method may further comprise actively exciting the solid object in dependence on the determined correlated evolution of the state of the solid object. The method may be used to find out the effect of a specific active excitation of the solid object. Reversely, the method may be used to find a specific active excitation of the solid object that leads to a desirable correlated evolution of the state of the solid object. Subsequently, this evolution may be triggered by actually applying the specific active excitation to the solid object. Examples of such active excitation may include, for example, exposure to a light or to an electric current or voltage.

Determining the closed set of hierarchical equations may comprise performing an averaging procedure, such as an ensemble averaging procedure, to a master equation that defines a time evolution of a probability that the solid object with its excitations is in a certain state in a configuration space. This averaging procedure allows to determine the set of hierarchical equations from a master equation.

The method may further comprise determining the master equation based on the plurality of properties of the solid object.

The approximation used to close the set of hierarchical equations may be based on a mean of a product of at least two occupations, divided by a product of the mean of each of said at least two occupations. This provides a particularly accurate and effective approximation.

The approximation may comprise a pair approximation (PA). This provides an accurate and efficient approximation.

The approximation may comprise a superposition approximation (SA). This provides an even more accurate, and still efficient approximation.

The superposition approximation (SA) may be performed at a 3rd order of accuracy by taking into account covariances of up to 3 occupations and neglecting covariances beyond 3 occupations. It was found that this may provide a good balance between accuracy and computational resources.

The superposition approximation (SA) may be performed at an order of accuracy higher than 3 by taking into account at least one covariance beyond 3 occupations. This provides a particularly accurate result.

The solid object may be composed of a plurality of different materials. For example, the solid object may be laminated. Alternatively, the solid object may be made of a composition of different materials. This allows to model practical situations.

The solid object may comprise a semiconducting material of an electronic device. The present method may be particularly suitable for analyzing semiconducting materials of an electronic device.

The solid object may form at least part of an organic electronic device. For example, the solid object may comprise an organic light emitting diode.

The solid object may comprise at least one of: a light emitting diode, a photovoltaic semiconductor, a photodetector, a transistor, a sensor, and at least part of a battery.

The dynamics of interacting excitations represented by the set of hierarchical equations may comprise interactions between excitations. For example, the interactions may comprise interactions between at least two excitons. Alternatively, the interactions may comprise interactions between an exciton and a charged particle. Herein, the charged particle may be, for example, a polaron. Yet alternatively, the interactions may comprise interactions between at least two charged particles. Herein, the charged particles may be polarons. In certain embodiments, combinations of the above types of interactions may also be represented by the set of hierarchical equations.

The interaction between the excitations may comprise exciton-exciton annihilation or exciton-polaron quenching.

The person skilled in the art will understand that the features described above may be combined in any way deemed useful. Moreover, modifications and variations described in respect of the system may likewise be applied to the method and to the computer program product, and modifications and variations described in respect of the method may likewise be applied to the system and to the computer program product.

### BRIEF DESCRIPTION OF THE DRAWINGS

In the following, aspects of the invention will be elucidated by means of examples, with reference to the drawings. The drawings are diagrammatic and may not be drawn to scale. Throughout the drawings, similar items may be marked with the same reference numerals. Reference is made to the following drawings:

FIG. 1. Sketch of the effect of correlations on triplet exciton positions in the presence of triplet-triplet annihilation, wherein FIG. 1A shows triplet excitons with random positions marked by points, as occurring at the beginning of an experiment under continuous illumination or in a steady state situation in the presence of strong exciton diffusion. FIG. 1B shows triplet excitons with correlated positions occurring as a result of a triplet-triplet annihilation (TTA) with no or weak diffusion in a steady state situation.

FIG. 2. Comparison of approximation methods and validation against KMC results: transient triplet exciton density in a simulation of a transient photoluminescence experiment in the absence of triplet diffusion.

FIG. 3. Comparison of approximation methods and validation against KMC results transient triplet exciton density at different initial triplet densities in the absence of triplet diffusion.

FIG. 4. Comparison of the approximation methods and validation against KMC results: triplet exciton pair correlation function g(r, t) in the absence of triplet diffusion.

FIG. 5. Comparison of the approximation methods and validation against KMC results steady-state triplet exciton density in the absence of triplet diffusion.

FIG. 6. Comparison of the approximation methods and validation against KMC results: steady-state triplet pair correlation functions g(r) in the absence of triplet diffusion.

FIG. 7. Comparison of the approximation methods and validation against KMC results: transient triplet exciton density in two dimensions in the absence of triplet diffusion.

FIG. 8. Comparison of the approximation methods and validation against KMC results: transient triplet exciton density in the presence of triplet diffusion.

FIG. 9. Comparison of the approximation methods and validation against KMC results: transient triplet pair correlation functions g(r, t) in the presence of triplet diffusion.

FIG. 10. Comparison of the approximation methods and validation against KMC results: steady-state triplet exciton density in the presence of triplet diffusion.

FIG. 11. Comparison of approximation methods and validation against KMC results: steady-state triplet exciton pair correlation function g(r) in the presence of diffusion.

FIG. 12. Flow chart illustrating aspects of a method of efficiently and accurately determining the dynamics of excitations in a material.

### DETAILED DESCRIPTION OF EMBODIMENTS

Certain exemplary embodiments will be described in greater detail, with reference to the accompanying drawings.

The matters disclosed in the description, such as detailed construction and elements, are provided to assist in a comprehensive understanding of the exemplary embodiments. Accordingly, it is apparent that the exemplary embodiments can be carried out without those specifically defined matters. Also, well-known operations or structures are not described in detail, since they would obscure the description with unnecessary detail.

The method and system disclosed herein may be used, for example, for molecular-scale simulations of opto-electronic processes in disordered systems, such as organic light emitting diodes (OLEDs), organic photovoltaics (OPV), and organic field-effect transistors (OFETs).

ME modelling of organic (opto) electronic devices may be done using a mean field approximation (MFA), where spatial correlations among excitations are neglected. This makes ME modelling computationally efficient but also inaccurate as compared to exact but expensive modelling techniques like KMC.

In the context of organic (opto) electronic devices, the excitations are the charges and/or excitons located on different molecules of a material. The mean field approximation (MFA) involves neglecting all spatial correlations among these interacting excitations.

The time evolution of the probabilities for a system of interacting excitations to be in the system's different quantum states can be mapped onto a hierarchical system of coupled equations for the spatial correlations functions of the excitations with increasing order. These are called Boguliobov-Born-Green-Kirkwood-Yvon (BBGKY) equations and provide a method to include spatial correlation effects up to any order of accuracy.

However, reaching a sufficient accuracy to satisfy the purpose of predicting the behavior of (opto) electronic devices by performing simulations of the system excitations is difficult in practice. This is because the complexity of the equations rapidly grows with increasing accuracy (order of approximation) and it quickly becomes impractical to find their solutions. Results obtained with the conventional BBGKY method up to the third order of approximation are shown to deviate significantly from KMC results. Higher orders of approximation to better describe the dynamics of the excitations that are relevant to simulate (opto) electronic and battery devices would become impractical due to e.g. computational resource constraints.

The method of the present disclosure solves this problem by the application of better approximations, based on a mean of a product of at least two occupations, divided by a product of the mean of each of said at least two occupations. Examples of such approximations include the pair approximation (PA) and the superposition approximation (SA). These approximations allow one to overcome the current limitations for obtaining a level of accuracy that is close to the accuracy achieved by KMC methods, with the advantage of having a computational time-frame that is practical for the engineer. This way, it becomes feasible to analyze more complex structures and stimuli, so that the number of prototype iterations in hardware design may be greatly reduced.

The PA is a low-excitation density approximation where the higher order correlation terms are ignored by taking the limit of excitation density to zero to provide a closure for the hierarchical system of equations.

The SA approximates the correlation function of a group of three excitations as a product of pair correlation functions. The excitations in the system are first considered as a collection of independently interacting excitation pairs, the spatial correlation function of all the possible interacting pairs (pair correlation function) is then calculated. Finally, the correlation function of three excitations is computed from the product of the pair correlation functions so that the system of hierarchical equations is closed.

The SA is the more accurate approximation of the two, with a computation time that is longer than that of the PA, but still shorter than KMC computation time.

In this detailed description, the proposed approximations will be explained in greater detail by means of the examples of PA and SA. However, the approximations are not limited to PA and SA, but can be extended to similar methods using, for example, a mean of a product of at least two occupations, divided by a product of the mean of each of said at least two occupations. Moreover, the techniques disclosed herein are illustrated using the detailed example of a prototypical case relevant for phosphorescent OLEDs, namely the mutual annihilation by a Förster type interaction of triplet excitons residing on phosphorescent organo-metallic guest molecules embedded in an organic fluorescent host material, a process known as triplet-triplet annihilation. However, the method of the invention is applicable to both electronic and excitonic excitations within the material, and provides sufficient accuracy to model such excitations in electronic, opto-electronic and battery devices.

The results obtained with PA and SA are benchmarked against KMC simulations and are found to be very accurate, while consuming much less computational resources.

According to certain embodiments, the method and system disclosed herein may allow one to simulate the interplay between all electronic and excitonic processes in OLEDs, OPV and OFETs, at the molecular scale, in all three dimensions, and from the nanosecond timescale to the full device lifetime. The simulations may predict the electrical characteristics, efficiency, color point, and/or lifetime of devices, based on physically meaningful material parameters, which may be obtained, for example, from dedicated experiments or from quantum chemistry. The performance of any combination of materials and stack architectures, under different operational conditions, can be predicted.

According to certain embodiments, the method and system disclosed herein may allow to predict electrical characteristics, efficiency, color point, and/or lifetime of devices, based on material parameters obtained from, for example, dedicated experiments or from quantum chemistry. By building a database of materials, the performance of any combination of these materials can be predicted.

According to certain embodiments, a solid object, such as an organic device, may be modeled as a 3D molecular-scale mechanistic device model. Any 3D structure is possible, including graded concentration profiles, laterally non-uniform structures, rough, intermixed interfaces. Information may be provided regarding a mixed-matrix emissive layer. This mechanistic device model or 3D structure may be indicative of a plurality of properties of the solid object, wherein the solid object comprises at least one material, and the plurality of properties comprise material properties of said at least one material, and may also comprise dimensions and information about interfaces between layers of different material.

According to certain embodiments, the correlated evolution of the state of the solid object may be determined by the method or system. This output may be analyzed to detect, for example, effects of disorder and traps, non-uniform currents, charge accumulation, charge carrier loss, or voltage loss across transport layers.

In certain embodiments, the output of the method, such as information based on the evolution of the state of the solid object, may be visualized. For example, the method may output information about current density filaments, events, charge and exciton movement, exciton generation and dissociation, exciton quenching, or degradation.

Using the techniques disclosed herein, it is possible to determine and account for the spatial correlations among excitations within a physical system (the system) defined by a material or device, such that the results of this determination of the correlations may be applied to obtain an accurate description and ultimately an accurate prediction of the system behavior.

The evolution of excitations that are predicted using the method disclosed herein, may be used to select suitable compositions of materials, dimensions, and geometry of electronic devices, and select suitable ways to operate them by triggering certain excitations by performing a stimulus. The selected options may then be implemented in electronic devices that are under development.

The techniques disclosed herein are directed primarily at an analysis of a (imaginary or real) solid object and its response to an applied stimulus. This solid object may in particular be composed of electronic materials, such as organic materials. The stimulus triggers excitations within the solid object, and these excitations have an impact on the electronic functioning of the electronic materials.

The techniques disclosed herein make it feasible to perform simulations of new classes of devices. In particular, simulations of light emitting diodes, photovoltaic semiconductors, photodetectors, transistors, sensors, neuromorphic devices, and batteries become feasible to do with an accuracy that approaches the accuracy of kinetic Monte Carlo simulations.

An aspect provides a computer-implemented method of determining a correlated evolution of a state relating to a solid object subjected to a stimulus, the method comprising obtaining information indicative of a composition and geometry of the solid object and obtaining information indicative of an external stimulus in respect of the solid object. This information may be received as input data, for example by means of a user interface or data file.

The method may further comprise determining a closed set of hierarchical equations representing dynamics of excitations within the solid object, including spatial correlations among the plurality of excitations, based on the composition and geometry of the solid object, wherein the hierarchical equations are indicative of a time evolution of correlation functions of the excitations, wherein the closed set of hierarchical equations is closed according to an approximation. For example, a (non-closed) set of hierarchical equations may be identified in dependence on the input data. This (non-closed) set may be closed by means of the approximations.

The method may further comprise determining the correlated evolution of the state of the solid object based on the closed set of hierarchical equations and the external stimulus. The closed set of equations may be solved numerically, for example.

The solid object may be an organic semiconductor device.

The method may further comprise predicting a physical behavior of the solid object based on the correlated evolution of the state of the solid object, wherein the physical behavior is associated with at least one of light emitted by the solid object, light absorbed by the solid object, energy stored within the solid object, energy absorbed by the solid object and energy released by the solid object. This physical behavior may be determined based on a predetermined mapping between the evolving profile of excitations and the relevant physical behavior.

The method may further comprise performing a measurement in respect of the solid object to generate at least part of the information. This measurement may be performed by means of a measurement device. The computer-implemented method may receive an outcome of an actual physical measurement using a measurement device in conjunction with an experiment on an actual physical object.

In certain embodiments, determining the closed set of hierarchical equations comprises performing an averaging procedure, such as an ensemble averaging procedure, to a master equation that defines a time evolution of a probability that the solid object with its excitations is in a certain state in a configuration space. The master equations, based on the composition and geometry of the solid object, may be determined in a way known to the person skilled in the art, in view of the present disclosure.

The approximation can be based on a mean of a product of at least two occupations, divided by a product of the mean of each of said at least two occupations. Specific examples are given in this detailed description, for example, in Equations (17) and (18). That approximation can include, for example, a pair approximation (PA) or a superposition approximation (SA).

The superposition approximation (SA) can be performed at a 3rd order of accuracy by taking into account covariances of up to 3 occupations and neglecting covariances beyond 3 occupations. Specifically, the superposition approximation (SA) can be performed at a 3rd order of accuracy by taking into account covariances of occupations at up to 3 sites and neglecting covariances of occupations at more than 3 sites.

The superposition approximation (SA) can alternatively be performed with greater accuracy at an order of accuracy higher than 3 by taking into account at least one covariance beyond 3 occupations. For example, all covariances of up to k occupations can be taken into account while neglecting all covariances of more than k occupations, wherein k is an integer greater than 3.

The solid object is composed of a plurality of different materials. The composition can thus involve more than one material. For example, the composition may specify volume % or mass % of each material in the composition. Moreover, different layers, blocks, or other regions of different materials or compositions may be joined together to form the solid object. This may be specified by the geometric information.

The solid object may comprise a semiconducting electronic device. For example, the composition of which the solid object is made may comprise a semiconducting material.

The solid object may form at least part of an organic electronic device. The methods disclosed herein may be advantageously performed for predicting physical behaviors of objects made of organic electronic materials, such as a light emitting diode, a photovoltaic semiconductor, a photodetector, a transistor, a sensor, and certain components of a battery.

The excitations can be at least two excitons. Alternatively, the excitation can be an exciton and a charged particle (polaron). Alternatively, the excitation can be at least two charged particles (polarons). The interactions can comprise at least one of exciton-exciton annihilation and exciton-polaron quenching. In a specific example, which is elaborated further below, the excitons are triplets, so that the interactions comprise at least one of triplet-triplet annihilation and triplet-polaron quenching.

A method of manufacturing a solid object with a particular physical behavior may comprise applying the computer-implemented method set forth above to determine the correlated evolution of the state of the solid object for a particular composition and geometry and external stimulus. Based on the determined evolution, which may be calculated in respect of an imaginary solid object, it may be decided to manufacture such a solid object. Thus, the method may further comprise manufacturing the solid object in dependence on the determined correlated evolution of the state of the solid object.

For example, the computer-implemented method set forth herein may be repeated in respect of a plurality of different combinations of composition and geometry, and the solid object to be manufactured may be selected based on the determined correlated evolution of the state of the solid object associated with each combination of composition and geometry. For example, by comparing the determined evolution to a certain desirable evolution.

A method of selecting a stimulus to a solid object may comprise repeatedly applying the computer-implemented method set forth herein in respect of a plurality of different stimuli, and selecting a stimulus for the solid object, based on the determined correlated evolution of the state of the solid object associated with each of the plurality of different stimuli. This allows one to control the physical behavior by applying a stimulus of which the effect has been determined beforehand using the computer-implemented method.

A system may be provided for determining a correlated evolution of a state relating to a solid object. The system may comprise at least one processor and a non-transitory computer readable media. The media may store instructions of a computer program that causes the at least one processor, when executing the instructions, to perform the steps of the method as elaborated herein, for example obtaining information indicative of a composition and geometry of the solid object; obtaining information indicative of an external stimulus in respect of the solid object; determining a closed set of hierarchical equations representing dynamics of excitations within the solid object, including spatial correlations among the plurality of excitations, based on the composition and geometry of the solid object, wherein the hierarchical equations are indicative of a time evolution of correlation functions of the excitations, wherein the closed set of hierarchical equations is closed according to an approximation; and determining the correlated evolution of the state of the solid object based on the closed set of hierarchical equations and the external stimulus.

### I. Introduction

The efficiency of organic light-emitting diodes (OLEDs) has dramatically increased by the introduction of phosphorescent emitters. In these emitters, the spin-orbit coupling induced by a heavy metal atom in the core of the molecule allows fast intersystem crossing (ISC) of singlet to triplet excitons and radiative decay of triplet excitons. As a result, all excitons formed can in principle contribute to phosphorescent emission. At low current densities, the internal quantum efficiency (IQE) of phosphorescent OLEDs approaches 100%. At high current densities, efficiency roll-off occurs, which is mainly attributed to a triplet-triplet annihilation (TTA) and a triplet-polaron quenching (TPQ). Due to these processes, correlations between the positions of surviving triplet excitons (TTA) and between the positions of charges and surviving triplet excitons (TPQ) will arise. This complicates the evaluation of the effects of TTA and TPQ on the photophysics of phosphorescent OLEDs and therefore their mitigation. Kinetic Monte Carlo simulations provide a mechanistic and therefore in principle exact way to evaluate these effects, but are relatively computationally expensive.

Recently, Shumilin and Beltukov addressed the problem of the influence of correlations in the positions of charges on single-carrier transport in molecular semiconductors (“System of correlation kinetic equations and the generalized equivalent circuit for hopping transport”, Physical Review B 100, 014202, 2019, hereafter “the approach of Shumilin and Beltukov”). Such correlations result from the strong Coulomb repulsion of two like charges on the same molecule, which makes the double occupancy very unlikely. Correlations in the occupancies of different molecular sites by charges exist even if the Coulomb repulsion of charges residing on different molecules is ignored due to the spatial correlation between position of the charges. The approach of Shumilin and Beltukov is based on an approximate numerical solution of the hierarchical Bogoliubov-Born-Green-Kirkwood-Yvon (BBGKY) chain of equations, which is equivalent to a master equation formulation of a system of interacting particles. In this hierarchical chain, the equation of motion for the n-particle distribution function has a dependency on the (n+1)-particle distribution function. Solution therefore requires a closure, which is obtained in the approach of Shumilin and Beltukov by taking into account covariances in the occupation of molecular sites by charges up to a certain order and neglecting higher-order covariances. In this way, the infinite system of hierarchical equations is truncated to a finite system, which can be solved numerically.

BBGKY-like chains of equations can also be formulated for and applied to the TTA and TPQ processes, but to the best of our knowledge this has not yet been done. Here, we choose to focus on TTA because of the presence of only one instead of two particle species, namely triplet excitons, or, for short, triplets. We will assume that TTA is a long-range Förster type process with an r₋₆ distance dependence between two triplets on phosphorescent guest molecules embedded in a host. In this process the excitation energy of one of the triplets is transferred to the molecule carrying the second triplet. The Forster type process that makes this transfer possible is based on the small amount of singlet character mixed in to triplet excitons on the phosphorescent emitter by the spin-orbit coupling that facilitates the phosphorescent emission. The acceptor is now in a highly excited triplet state and will relax thermally to the lowest triplet state, by which the energy of the donor triplet is lost. TTA can also occur by a Dexter type process, but this is only important at very high guest concentrations, that are not relevant for efficient phosphorescent OLEDs because of concentration quenching.

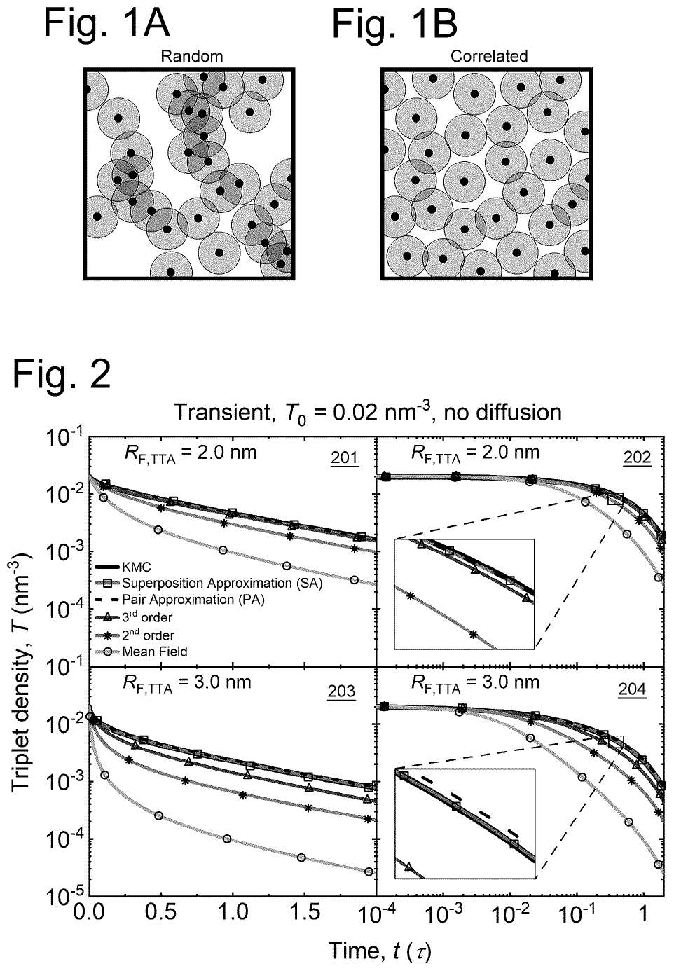

FIG. 1 displays two-dimensional sketches of a system with random positions of the triplets (FIG. 1A) and a system with correlated triplet positions (FIG. 1B) that has undergone TTA. Such a situation occurs, for example, in an experiment under continuous illumination where triplets are generating at random positions by a illumination, after which they disappear either by radiative decay or TTA. A diameter of the circles drawn around each triplet is a measure for the decay length of the two-exciton, or pair, correlation function and the region inside the circle signifies a triplet depletion zone. A proper description of the resulting structure of positionally correlated surviving triplets requires a many-body treatment. Apart from TTA, we will consider triplet diffusion among the emitters and assume that this diffusion is also a Förster type process. Diffusion of triplets decreases the correlation established by TTA. Sufficiently strong diffusion will wash out correlations completely.

Models for TTA often ignore the positional correlations of triplet excitons, describing the decay of the time-dependent triplet density T(t) in a TRPL experiment by the phenomenological equation

$$\frac{dT}{dt} = - {k}_{r} T - \frac{1}{2} {k}_{TTA} {T}^{2} , \qquad ( 1 )$$

where kᵣ is the radiative triplet decay rate and k_{TTA} a TTA rate coefficient. It has been shown from KMC simulations that such a description is inaccurate, because it neglects correlations in the positions of surviving triplets. Instead, two effective TTA rate coefficients were introduced to describe a TRPL experiment: a rate coefficient k_{TTA,1} based on the measured time at which half of the emission has taken place and a rate coefficient k_{TTA,2} based on the decrease of the total, integrated, photoluminescence (PL) efficiency with respect to the zero fluence limit, in which TTA is absent. The ratio r≡k_{TTA,2}/k_{TTA,1} is equal to 1 in the strong-diffusion limit, where Eq. 1 is valid, but can exceed 2 in the absence of triplet diffusion.

In the present disclosure, we evaluate the effects of TTA in phosphorescent host-guest systems by master equation modeling, including correlations in the triplet positions. Our approach, described in Sec. II, starts by formulating the master equation for the probabilities that the system of triplets is in a particular state. We then derive from this master equation a hierarchy of equations for the n-triplet distribution functions. Following the approach of Shumilin and Beltukov we consider a closure of the hierarchy by neglecting covariances beyond a certain order, where we in particular consider the 2^{nd} and 3^{rd} order approximation. We find, however, that these approximations are not sufficiently accurate. Much more accurate results are obtained from a low-triplet density, or pair, approximation, (PA) and from an approximation known as the superposition approximation (SA).

### II. Methods

In this section, we discuss the theoretical and computational methods used in our calculations of TTA. The system we study represents an emission layer of a phosphorescent OLED. It consists of a cubic lattice of sites, with a fraction c_{g} of randomly positioned guest sites in an environment of host sites. The guest sites represent phosphorescent molecules that can carry triplet excitons. The host sites are assumed to be inaccessible to the excitons. For the lattice constant we take a=1 nm, which is a typical value for the intermolecular distance in molecular semiconductors. We neglect energetic disorder in this discussion, so that there is no energy difference between triplet excitons at different guest sites. Explicit calculations for Ir-cored phosphorescent emitters yield an approximately Gaussian triplet energy disorder with a standard deviation of about 0.05 eV. We consider the following processes:
(1) Radiative decay of triplet excitons on the guest sites with a rate kᵣ=1/T, where T is the radiative triplet exciton lifetime, which is taken to be equal for all sites. We assume here that the non-radiative decay rate is zero, but it is straightforward to extend the present results to a non-zero radiative decay rate.
(2) TTA by a Förster process governed by a Förster radius R_{F,TTA}, where one of the two triplet excitons involved in the process is annihilated.
(3) Diffusion of triplet excitons between guest sites by a Förster process governed by a Förster radius R_{F,diff}.
(4) Generation of excitons with a generation rate Gat sites that are not yet occupied by a triplet exciton. This generation can take place by illumination, such as in a PL experiment, or by recombination of electrons and holes. We will consider two different situations, corresponding to two types of experiments. In the first situation, we start with a randomly generated configuration of triplet excitons on guest sites and follow the density T(t) of excitons as a function of time t. This situation is representative of a TRPL experiment, where a phosphorescent emission layer is illuminated by a short laser pulse, after which the luminescence is measured as a function of time. In the second situation, there is a constant generation rate G of triplet excitons and the steady-state density of excitons T as a function of G is studied. This situation is representative of an experiment under continuous illumination or to a condition where excitons are homogeneously generated by electron-hole recombination in an emission layer of an OLED.

### A. Kinetic Monte Carlo Simulations

Our benchmark results are obtained with kinetic Monte Carlo (KMC) simulations, performed with the software tool Bumblebee (available at https://simbeyond.com, the Bumblebee software is provided by Simbeyond B. V.). Simulation boxes of 50×50×50 sites are used with periodic boundary conditions. The rate Sᵢⱼ for TTA involving two triplet excitons at positions i and j at a mutual distance rᵢⱼ, annihilating the triplet exciton at i, is given by:

$${S}_{ij} = S ( {r}_{ij} ) = \frac{1}{τ} ( \frac{{R}_{F , TTA}}{{r}_{ij}} \text{?} . \qquad ( 2 )$$

$$\text{?} \text{indicates text missing or illegible when filed}$$

The rate Dᵢⱼ for the Förster transfer of a triplet exciton at i to j is given by

$${D}_{ij} = D ( {r}_{ij} ) = \frac{1}{τ} ( \frac{{R}_{F , diff}}{{r}_{ij}} \text{?} . \qquad ( 3 )$$

$$\text{?} \text{indicates text missing or illegible when filed}$$

We impose cutoff distances 2 R_{F,TTA} and 2 R_{F,diff} on the TTA and diffusion processes beyond which the rates of these processes are taken zero. We checked that taking larger cutoffs has no significant influence on the results presented in this work. Since we neglect energy disorder, the TTA and diffusion rates are symmetric: Sᵢⱼ=Sⱼᵢ and Dᵢⱼ=Dⱼᵢ. In the presented results, we take averages over 50-300 different simulation runs, depending on the required accuracy. Standard deviation on the results are indicated when appropriate.

### B. General Theory

The state of the system is fully specified by the occupations nₖ ∈{0, 1} of all N guest sites by triplet excitons. In a master equation approach, one considers the transition rates between all the possible states of the system. The time dependence of the probability P_{ζ}(t)≡P (n₁, . . . , n_{N}; t) to be at time t in the state ζ which has the occupations (n₁, . . . , n_{N}), is given by the master equation

$$\frac{dP ( {n}_{1} , … , {n}_{N} ; t )}{dt} = {\sum }_{\text{?} \neq i} [ - {S}_{ij} {n}_{i} {n}_{j} P ( {n}_{1} , … , {n}_{N} ; t ) + {S}_{ij} {n}_{i} ( 1 - {n}_{j} ) P ( {n}_{1} , … , {n}_{i} , … , {n}_{j} + 1 , … , {n}_{N} ; t ) ] + {\sum }_{i , j , \text{?} \neq i} [ - {D}_{ij} {n}_{i} ( 1 - {n}_{j} ) P ( {n}_{1} , … , {n}_{N} ; t ) + {D}_{ij} {n}_{j} ( 1 - {n}_{1} ) P ( {n}_{1} , … , {n}_{i} + 1 , … , {n}_{j} - 1 , {n}_{N} ; t ) ] + {\sum }_{i} [ - {k}_{\text{?}} {n}_{\text{?}} P ( {n}_{1} , … , {n}_{N} ; t ) + {k}_{\text{?}} ( 1 - {n}_{i} ) P ( {n}_{1} , … , {n}_{i} + 1 , … , {n}_{N} ; t ) ] + {\sum }_{i} [ - G ( 1 - {n}_{i} ) P ( {n}_{1} , … , {n}_{N} ; t ) + {Gn}_{i} P ( {n}_{1} , … , {n}_{i} - 1 , … , {n}_{N} ; t ) ] . \qquad ( 4 )$$

$$\text{?} \text{indicates text missing or illegible when filed}$$

For realistic values of N it is impossible to directly solve these coupled equations for the 2^{N} probabilities Pζ(f). Performing KMC simulations is actually a way to solve these equations, but obtaining sufficient accuracy for large systems requires relatively long simulation times. Our alternative approach is based on deriving a chain of equations for the n-exciton distribution functions similar to the Bogoliubov-Born-Green-Kirkwood-Yvon (BBGKY) chain of equations. The notation we will use corresponds largely to that of Shumilin and Beltukov used in their approach. We start by defining the one-triplet distribution function

$$\overline{{n}_{k}} = {\sum }_{ξ} {n}_{k , ξ} {P}_{ξ} ( t ) , \qquad ( 5 )$$

where Σ_{ζ}P_{ζ}(t)=1, n_{k, ζ} is the occupation number of site k(0 or 1) in state ζ, and where we omit the t-dependence of n̅ₖ̅. From Eq. (4) in conjunction with Eq. (5) we can derive for n̅ₖ̅ the equation

$$\frac{d \overline{{n}_{k}}}{dt} = - {k}_{\text{?}} \overline{{n}_{k}} + G ( 1 - \overline{{n}_{k}} ) - {\sum }_{\text{?} \neq k} [ {S}_{k \text{?}} \overline{{n}_{k}} \overline{{n}_{\text{?}}} + {D}_{ki} ( \overline{{n}_{k}} - \overline{{n}_{\text{?}}} ) ] , \qquad ( 6 )$$

$$\text{?} \text{indicates text missing or illegible when filed}$$

Where n̅ₖ̅n̅₁̅ is the two-triplet distribution function

$$\overline{{n}_{k} {n}_{\text{?}}} = {\sum }_{ξ} {n}_{k , ξ} {n}_{\text{?} , ξ} {P}_{ξ} ( t ) . \qquad ( 7 )$$

$$\text{?} \text{indicates text missing or illegible when filed}$$

For the two-triplet distribution function we can subsequently derive the equation

$$\frac{d \overline{{n}_{k} {n}_{\text{?}}}}{dt} = - 2 {k}_{\text{?}} \overline{{n}_{k} {n}_{\text{?}}} - 2 {S}_{\text{?}} \overline{{n}_{k} {n}_{\text{?}}} + G ( \overline{{n}_{k}} + \overline{{n}_{\text{?}}} - 2 \overline{{n}_{k} {n}_{\text{?}}} ) - {\sum }_{m \neq k , \text{?}} ( {S}_{km} + {S}_{im} ) \overline{{n}_{k} {n}_{\text{?}} {n}_{\text{?}}} + {\sum }_{m \neq k , \text{?}} [ {D}_{km} ( \overline{{n}_{\text{?}} {n}_{m}} - \overline{{n}_{k} {n}_{\text{?}}} ) + {D}_{\text{?}} ( \overline{{n}_{k} {n}_{m}} - \overline{{n}_{k} {n}_{\text{?}}} ) ] , \qquad ( 8 )$$

$$\text{?} \text{indicates text missing or illegible when filed}$$

and for the three-triplet distribution function the equation

$$\frac{d \overline{{n}_{k} {n}_{\text{?}} {n}_{m}}}{dt} = - 3 {k}_{\text{?}} \overline{{n}_{k} {n}_{\text{?}} {n}_{m}} - 2 ( {S}_{k \text{?}} + {S}_{km} + {S}_{m \text{?}} ) \overline{{n}_{k} {n}_{\text{?}}} + G ( \overline{{n}_{k} {n}_{\text{?}}} + \overline{{n}_{k} {n}_{m}} + \overline{{n}_{\text{?}} {n}_{m}} - 3 \overline{{n}_{k} {n}_{\text{?}} {n}_{m}} ) - {\sum }_{p \neq k , i , m} ( {S}_{kp} + {S}_{\text{?} p} + {S}_{mp} ) \overline{{n}_{k} {n}_{\text{?}} {n}_{m} {n}_{p}} + {\sum }_{p \neq k , i , m} [ {D}_{kp} ( \overline{{n}_{\text{?}} {n}_{\text{?}} {n}_{p}} - \overline{{n}_{k} {n}_{\text{?}} {n}_{m}} ) + {D}_{ip} ( \overline{{n}_{k} {n}_{m} {n}_{p}} - \overline{{n}_{k} {n}_{\text{?}} {n}_{m}} ) + {D}_{mp} ( \overline{{n}_{k} {n}_{\text{?}} {n}_{p}} - \overline{{n}_{\text{?}} {n}_{\text{?}} {n}_{m}} ) ] . \qquad ( 9 )$$

$$\text{?} \text{indicates text missing or illegible when filed}$$

The complete chain of coupled equations for the n-triplet distribution functions of increasing n can be written down in the following form:

$$\frac{d \overline{{\prod }_{\text{?} ∈ \text{?}} {n}_{i}}}{dt} = - {k}_{r} \vert I \vert \overline{{\prod }_{\text{?} ∈ \text{?}} {n}_{i}} - 2 \overline{{\prod }_{\text{?} ∈ \text{?}} {n}_{i}} {\sum }_{i , j ∈ \text{?} , j > i} {S}_{ij} + G ( {\sum }_{\text{?} ∈ \text{?}} \overline{{\prod }_{\text{?} ∈ \text{?}} {n}_{j}} - \vert I \vert \overline{{\prod }_{\text{?} ∈ \text{?}} {n}_{i}} ) - {\sum }_{\text{?} ∈ \text{?}} {\sum }_{\text{?} ∈ \text{?}} ( {S}_{j \text{?}} \overline{{n}_{\text{?}} {\prod }_{\text{?} ∈ \text{?}} {n}_{i}} ) + {\sum }_{j ∈ \text{?}} {\sum }_{\text{?}} {D}_{j \text{?}} ( \overline{{n}_{\text{?}} {\prod }_{\text{?} ∈ \text{?}} {n}_{i}} - \overline{{\prod }_{\text{?} ∈ \text{?}} {n}_{i}} ) , \qquad ( 10 )$$

$$\text{?} \text{indicates text missing or illegible when filed}$$

where l={i₁, i₂, . . . , i_{/l/}} is an arbitrary set of /l/sites.

Following the approach of Shumilin and Beltukov we introduce the covariances

$$\begin{matrix}\overline{δ {n}_{I}} ≡ \overline{{\prod }_{\text{?} ∈ \text{?}} δ {n}_{i}} = \overline{{\prod }_{\text{?} ∈ \text{?}} ( {n}_{i} - \overline{{n}_{\text{?}}} )} \\ ≡ {\sum }_{J ∈ \text{?}} {( - 1 )}^{\vert \text{?} \vert - \vert J \vert} ( \overline{{\prod }_{\text{?} ∈ \text{?}} {n}_{i}} {\prod }_{\text{?} ∈ \text{?}} \overline{{n}_{\text{?}}} )\end{matrix} \text{?} \qquad ( 11 )$$

$$\text{?} \text{indicates text missing or illegible when filed}$$

which is an alternating sum over all subsets J⊂l. Neglecting covariances beyond m sites, by putting on; δnₗ=0 when |/|≥m+1, leads to a closure of Eqs. (10) that we will call the m-th order approximation. The mean field (MF), or 1^{st} order, approximation corresponds to m=1:

$$\overline{δ {n}_{k} δ {n}_{\text{?}}} = \overline{( {n}_{k} - \overline{{n}_{k}} ) ( {n}_{\text{?}} - \overline{{n}_{\text{?}}} )} = \overline{{n}_{k} {n}_{\text{?}}} - \overline{{n}_{k} {n}_{\text{?}}} = 0. \qquad ( 12 )$$

$$\text{?} \text{indicates text missing or illegible when filed}$$

With this, Eq. (6) transforms into

$$\frac{d \overline{{n}_{k}}}{dt} = - {k}_{\text{?}} \overline{{n}_{k}} + G ( 1 - \overline{{n}_{k}} ) - {\sum }_{\text{?} ∈ k} ( {S}_{k \text{?}} \overline{{n}_{k}} \overline{{n}_{\text{?}}} + {D}_{k \text{?}} ( \overline{{n}_{k}} - \overline{{n}_{\text{?}}} ) ) . \qquad ( 13 )$$

$$\text{?} \text{indicates text missing or illegible when filed}$$

In this work, we will show results obtained with the MF, 2^{nd}, and 3^{rd} order approximation. For the 2^{nd} order approximation we have

$$\overline{δ {n}_{k} δ {n}_{\text{?}} δ {n}_{m}} = \overline{( {n}_{k} - \overline{{n}_{k}} ) ( {n}_{\text{?}} - \overline{{n}_{\text{?}}} ) ( {n}_{m} - \overline{{n}_{m}} )} = \overline{{n}_{k} {n}_{\text{?}} {n}_{m}} - \overline{{n}_{k} {n}_{\text{?}} {n}_{m}} - \overline{{n}_{k} {n}_{\text{?}}} \overline{{n}_{\text{?}}} - \overline{{n}_{\text{?}} {n}_{m}} \overline{{n}_{k}} + 2 \overline{{n}_{k}} \overline{{n}_{\text{?}}} \overline{{n}_{m}} = 0. \qquad ( 14 )$$

$$\text{?} \text{indicates text missing or illegible when filed}$$

So, in the 2^{nd} order approximation we replace the three-triplet distribution function by lower-order triplet distribution functions:

$$\overline{{n}_{k} {n}_{\text{?}} {n}_{m}} \approx \overline{{n}_{k} {n}_{\text{?}} {n}_{m}} + \overline{{n}_{k} {n}_{m}} \overline{{n}_{\text{?}}} + \overline{{n}_{\text{?}} {n}_{m}} \overline{{n}_{k}} - 2 \overline{{n}_{k} {n}_{\text{?}}} \overline{{n}_{\text{?}}} . \qquad ( 15 )$$

$$\text{?} \text{indicates text missing or illegible when filed}$$

In the 3^{rd} order approximation we put δnₖδnₗδnₘδnₚ=0, obtaining

$$\overline{{n}_{k} {n}_{\text{?}} {n}_{m} {n}_{p}} = \overline{{n}_{k} {n}_{\text{?}} {n}_{m}} \overline{{n}_{p}} + \overline{{n}_{k} {n}_{\text{?}} {n}_{p}} \overline{{n}_{m}} + \overline{{n}_{k} {n}_{m} {n}_{p}} \overline{{n}_{\text{?}}} + \overline{{n}_{\text{?}} {n}_{m} {n}_{p}} \overline{{n}_{k}} - \overline{{n}_{k} {n}_{\text{?}} {n}_{m}} \overline{{n}_{p}} - \overline{{n}_{k} {n}_{m}} \overline{{n}_{\text{?}}} \overline{{n}_{p}} - \overline{{n}_{k} {n}_{p}} \overline{{n}_{\text{?}}} \overline{{n}_{m}} - \overline{{n}_{\text{?}} {n}_{m}} \overline{{n}_{k}} \overline{{n}_{p}} - \overline{{n}_{\text{?}} {n}_{p}} \overline{{n}_{k}} \overline{{n}_{m}} - \overline{{n}_{m} {n}_{p}} \overline{{n}_{k}} \overline{{n}_{\text{?}}} + 3 \overline{{n}_{k}} \overline{{n}_{\text{?}}} \overline{{n}_{m}} \overline{{n}_{p}} . \qquad ( 16 )$$

$$\text{?} \text{indicates text missing or illegible when filed}$$

Next to these approximations, we consider two other approximations, which will turn out to be more accurate than the 2^{nd} and 3^{rd} order approximations. The first is the pair approximation (PA), which is essentially a low-triplet exciton density approximation. The second is the superposition approximation (SA), where the three-site correlation function is approximated as a product of two-site correlation functions. We define the two-site, or pair, correlation function as

$${g}_{k \text{?}}^{( 2 )} = \frac{\overline{{n}_{k} {n}_{\text{?}}}}{\overline{{n}_{k}} \overline{{n}_{\text{?}}}} , \qquad ( 17 )$$

$$\text{?} \text{indicates text missing or illegible when filed}$$

and the three-site correlation function as

$${g}_{k \text{?} m}^{( 3 )} = \frac{\overline{{n}_{k} {n}_{\text{?}} {n}_{m}}}{\overline{{n}_{k}} \overline{{n}_{\text{?}}} \overline{{n}_{m}}} . \qquad ( 18 )$$

$$\text{?} \text{indicates text missing or illegible when filed}$$

With these definitions Eq. (6) becomes

$$\frac{d \overline{{n}_{k}}}{dt} = {k}_{r} \overline{{n}_{k}} + G ( 1 - \overline{{n}_{k}} ) - {\sum }_{l \neq k} ( \overline{{n}_{k}} \overline{{n}_{l}} {S}_{{klg}_{kl}^{( 2 )}} + {D}_{kl} ( \overline{{n}_{k}} - \overline{{n}_{l}} ) ) , \qquad ( 19 )$$

and Eq. (8) becomes, using Eq. (19),

$$\frac{{dg}_{kl}^{( 2 )}}{dt} = 2 {Sklg}_{kl}^{( 2 )} + G ( \frac{1}{\overline{{n}_{k}}} + \frac{1}{\overline{{n}_{l}}} ) ( 1 - {g}_{kl}^{( 2 )} ) \qquad ( 20 )$$

$$- {\sum }_{m \neq k , l} \overline{{n}_{m}} ( {S}_{km} + {S}_{km} ) {g}_{klm}^{( 3 )}$$

$$+ {g}_{kl}^{( 2 )} [ {\sum }_{m \neq k} \overline{{n}_{m}} {S}_{km} {g}_{km}^{( 2 )} + {\sum }_{m \neq k} \overline{{n}_{m}} {S}_{lm} {g}_{lm}^{( 2 )} ]$$

$$+ {g}_{kl}^{( 2 )} {D}_{kl} ( 2 - \frac{\overline{{n}_{l}}}{\overline{{n}_{k}}} - \frac{\overline{{n}_{k}}}{\overline{{n}_{l}}} )$$

$$- {g}_{kl}^{( 2 )} {\sum }_{m \neq k , l} \overline{{n}_{m}} ( \frac{1}{\overline{{n}_{k}}} {D}_{km} + \frac{1}{\overline{{n}_{l}}} {D}_{lm} )$$

$$+ {\sum }_{m \neq k , l} \overline{{n}_{m}} ( \frac{1}{\overline{{n}_{k}}} {D}_{km} {g}_{km}^{( 2 )} + \frac{1}{\overline{{n}_{l}}} {D}_{lm} {g}_{km}^{( 2 )} ) .$$

In the low-triplet density limit we can neglect in Eq. (20) the 3^{rd} and 4^{th} terms, because they are of higher order in the triplet density than the other terms, leading to

$$\begin{matrix}\frac{{dg}_{kl}^{( 2 )}}{dt} = - 2 {Sklg}_{kl}^{( 2 )} + G ( \frac{1}{\overline{{n}_{k}}} + \frac{1}{\overline{{n}_{l}}} ) ( 1 - {g}_{kl}^{( 2 )} ) \\ + {g}_{kl}^{( 2 )} {D}_{kl} ( 2 - \frac{\overline{{n}_{l}}}{\overline{{n}_{k}}} - \frac{\overline{{n}_{k}}}{\overline{{n}_{l}}} ) \\ - {g}_{kl}^{( 2 )} {\sum }_{m \neq k , l} \overline{{n}_{m}} ( \frac{1}{\overline{{n}_{k}}} {D}_{km} + \frac{1}{\overline{{n}_{l}}} {D}_{lm} ) \\ + {\sum }_{m \neq k , l} \overline{{n}_{m}} ( \frac{1}{\overline{{n}_{k}}} {D}_{km} {g}_{lm}^{( 2 )} + \frac{1}{\overline{{n}_{l}}} {D}_{lm} {g}_{km}^{( 2 )} ) .\end{matrix} \qquad ( 21 )$$

We call this the pair approximation (PA) because in the low-triplet density limit the situation with the lowest density where there is still TTA is that of a pair of two triplets. In the superposition approximation (SA) one assumes that the correlation between three particles (in our case triplets) arises from independent pair interactions (in our case the TTA process), so that the three-site correlation function gₖᵢₘ⁽³⁾ in Eq. (20) is approximated as gₖₗₘ⁽³⁾≈g_{k/}⁽²⁾ gₖₘ⁽²⁾ gₗₘ⁽²⁾. Using the SA in Eq. (20) yields:

$$\begin{matrix}\frac{{dg}_{kl}^{( 2 )}}{dt} = - 2 {S}_{{klg}_{kl}^{( 2 )}} + G ( \frac{1}{\overline{{n}_{k}}} + \frac{1}{\overline{{n}_{l}}} ) ( 1 - {g}_{kl}^{( 2 )} ) \\ - {g}_{kl}^{( 2 )} {\sum }_{m \neq k , l} \overline{{n}_{m}} ( {S}_{km} + {S}_{lm} ) {g}_{km}^{( 2 )} {g}_{lm}^{( 2 )} \\ + {g}_{kl}^{( 2 )} [ {\sum }_{m \neq k} \overline{{n}_{m}} {S}_{km} {g}_{km}^{( 2 )} + {\sum }_{m \neq l} \overline{{n}_{m}} {S}_{lm} {g}_{lm}^{( 2 )} ] \\ + {g}_{kl}^{( 2 )} {D}_{kl} ( 2 - \frac{\overline{{n}_{l}}}{\overline{{n}_{k}}} - \frac{\overline{{n}_{k}}}{\overline{{n}_{l}}} ) \\ - {g}_{kl}^{( 2 )} {\sum }_{m \neq k , l} \overline{{n}_{m}} ( \frac{1}{\overline{{n}_{k}}} {D}_{km} + \frac{1}{\overline{{n}_{l}}} {D}_{lm} ) \\ + {\sum }_{m \neq k , l} \overline{{n}_{m}} ( \frac{1}{\overline{{n}_{k}}} {D}_{km} {g}_{lm}^{( 2 )} + \frac{1}{\overline{{n}_{l}}} {D}_{lm} {g}_{km}^{( 2 )} ) .\end{matrix} \qquad ( 22 )$$

We can solve Eqs. (21) and (22) for gₖₗ⁽²⁾ in conjunction with Eq. (19) to get results in the PA and SA, respectively.

### C. No Triplet Diffusion

We first discuss the case without triplet diffusion, by putting Dₖₗ=0 in the equations of the previous subsection. If there is no diffusion and the guest sites are randomly distributed, we do not need to distinguish between guest and host sites. The system then has translational symmetry and we can put n̅ₖ̅=n̅₀̅≡n̅(̅t̅)̅ for all k, where k=0 is the site at the origin. We are interested in calculating the triplet exciton density T(t)=n(t)/a³, which can be obtained by transforming Eq. (19) into

$$\frac{dT}{dt} = {k}_{r} T + G ( \frac{1}{{a}^{3}} - T ) - {T}^{2} {a}^{3} {\sum }_{k \neq 0} S ( {r}_{0 l} ) g ( {r}_{0 l} , t ) , \qquad ( 23 )$$

$$g ( {r}_{kl} , t ) ≡ {g}_{kl}^{( 2 )} , \qquad ( 24 )$$

where we make use of the fact that the pair correlation function now only depends on the distance between the two sites.

We start by considering a transient situation with no generation of excitons, G=0, describing a TRPL experiment. At time t=0 we have a density T₀ of randomly placed triplet excitons with uncorrelated positions. The MF expression for T is obtained by neglecting correlations and is equivalent to Eq. (1). It follows by putting g(r₀ₗ, t)=1 in Eq. (23):

$$\frac{dT}{dt} = - {k}_{r} T - \frac{1}{2} {k}_{TTA} {T}^{2} , \qquad ( 25 )$$

with the MF TTA rate coefficient defined by

$${k}_{TTA} ≡ 2 \times 8.402 {k}_{r} \frac{{R}_{F}^{6} , TTA}{{a}^{3}} , \qquad ( 26 )$$

where we performed the three-dimensional (3D) lattice sum

$${a}^{6} {\sum }_{k \neq 0} \frac{1}{{r}_{0 l}^{6}} = 8.402 . \qquad ( 27 )$$

Equation (25) can be solved analytically, yielding

$$T ( t ) = \frac{{k}_{r} {T}_{0}}{( {k}_{r} + \frac{1}{2} {k}_{TTA} {T}_{0} ) exp ( {k}_{r} t ) - \frac{1}{2} {k}_{TTA} {T}_{0}} . \qquad ( 28 )$$

We will also consider the two-dimensional (2D) equivalent of these results. The 2D equivalent of the lattice sum in Eq. (27) has the value 4.659. Equation (25) remains the same, where Tis now expressed in nm⁻² with k_{TTA}≡2×4.659R⁶_{F,TTA}/a⁴.

In the 2^{nd} order approximation we solve Eqs. (6) and (8), using the approximation Eq. (15) in the latter equation. In the 3^{rd} order approximation we include Eq. (9) in addition to Eqs. (6) and (8), and use the approximation Eq. (16). In both cases we have an infinite system of coupled time-dependent differential equations, which we cut off to a finite system by only considering TTA for sites separated by a distance smaller than 2 R_{F,TTA}, like in the KMC simulations. Equivalently, we replace distribution functions of 2^{nd} and 3^{rd} order by lower-order distribution functions when two sites are further apart than 2 R_{F,TTA}, where the 1^{st} order distribution function is n(t). We checked that with this procedure sufficient accuracy is obtained in the presented results. We exploit the point group symmetry of the cubic lattice to further reduce the number of equations. The resulting finite system of coupled time-dependent differential equations is solved with standard numerical techniques, using as boundary conditions at t=0 uncorrelated distribution functions.

Turning to the pair approximation, Eq. (21) becomes, putting Dₖₗ=G=0 and using Eq.

(24),

$$\frac{dg ( {r}_{0 l} , t )}{dt} = - 2 S ( {r}_{0 l} ) g ( {r}_{0 l} , t ) , \qquad ( 29 )$$

which leads, with g(r₀ₗ, t=0)=1, to

$$g ( {r}_{0 l} , t ) = exp [ - 2 {k}_{r} {t ( \frac{{R}_{F , TTA}}{{r}_{0 l}} )}^{6} ] . \qquad ( 30 )$$

Inserting this into Eq. (23), we obtain, with G=0:

$$\frac{dT}{dt} = - {k}_{r} T - {T}^{2} {a}^{3} {\sum }_{l \neq 0} S ( {r}_{0 l} ) g ( {r}_{0 l} , t ) . \qquad ( 31 )$$

In very good approximation, we can replace the lattice sum in this equation by an integral, which can be performed analytically:

$${\sum }_{l \neq 0} S ( {r}_{0 l} ) g ( {r}_{0 l} , t ) \approx \qquad ( 32 )$$

$$\frac{{k}_{r}}{{a}^{3}} \int {dr ( \frac{{R}_{F , TTA}}{r} )}^{6} exp [ - 2 {{tk}_{r} ( \frac{{R}_{F , TTA}}{r} )}^{6} ]$$

$$= \frac{\sqrt{2}}{3} {{π}^{\frac{3}{3}} ( \frac{{R}_{F , TTA}}{a} )}^{3} \sqrt{\frac{{k}_{r}}{t}} .$$

With this, Eq. (31) can be solved, resulting in

$$T ( t ) = \frac{{T}_{0} {e}^{- {k}_{r} t}}{1 + \frac{\sqrt{2}}{3} {π}^{2} {R}_{F , TTA}^{3} {T}_{0} erf ( \sqrt{{k}_{r} t} )} . \qquad ( 33 )$$

This result is equal to Eq. (17) in work of Engel et al. (“Ultrafast relaxation and exciton-exciton annihilation in PTCDA thin films at high excitation densities”, Chemical physics 325, 170, 2006), with the replacement ⅓→\sqrt{2}/3 in the denominator of that equation, and to Eq. (5) in work of Coehoorn et al. (“Effect of exciton diffusion on the triplet-triplet annihilation rate in organic semiconductor host-guest systems”, Physical Review B 99, 024201, 2019) with b=1/\sqrt{2}. Using the 2D equivalent of the integral in Eq. (32), we get

$${\sum }_{l \neq 0} S ( {r}_{0 l} ) g ( {r}_{0 l} , t ) \approx \frac{πΓ ( \frac{2}{3} )}{3 {2}^{2 / 3}} {( \frac{{R}_{F , TTA}}{a} )}^{2} \frac{{k}_{r}^{1 / 3}}{{t}^{2 / 3}} , \qquad ( 34 )$$

yielding the following 2D equivalent of Eq. (33) for the PA transient

$$T ( t ) = \frac{\text{?}}{1 + \frac{\text{?}}{\text{?}} {T}_{0} \text{?} π Γ ( \frac{2}{3} ) ( Γ ( \frac{1}{3} ) - Γ ( \frac{1}{3} \text{?} ) )} \text{?} \qquad ( 35 )$$

$$\text{?} \text{indicates text missing or illegible when filed}$$

with Γ(z) the Euler gamma function and Γ(b,z) the upper incomplete gamma function.

Finally, in the superposition approximation we obtain from Eq. (22)

$$( 36 )$$

$$\frac{dg ( \text{?} , t )}{dt} = - 2 S ( \text{?} ) g ( \text{?} , t ) - T \text{?} ( S ( \text{?} ) + S ( \text{?} ) ) g ( \text{?} , t ) g ( \text{?} , t ) g ( \text{?} , t ) + 2 T \text{?} g ( \text{?} , t ) \text{?} S ( \text{?} ) g ( \text{?} , t ) \text{?}$$

$$\text{?} \text{indicates text missing or illegible when filed}$$

Numerically solving this equation in conjunction with Eq. (23) yields the SA results. The numerical procedure we used is similar to that used for the 2^{nd} and 3^{rd} order approximation.

We now consider the steady state situation, where we are interested in the steady state triplet exciton density T as a function of the triplet generation rate G. The MF result is obtained by putting dT/dt=0 and g(r₀ₗ, t)=1 in Eq. (23), which leads to

$$T ( G ) = \frac{\sqrt{{( G + \text{?} )}^{2} + 2 G \text{?} / \text{?}} - ( G + \text{?} )}{\text{?}} \text{?} \qquad ( 37 )$$

$$\text{?} \text{indicates text missing or illegible when filed}$$

The steady state results in the 2^{nd} and 3^{rd} order approximation are obtained in the same way as in the transient case, but now with the inclusion of the terms containing G. We numerically solve the coupled system of time-dependent differential equations until no further change in time is observed. The results are independent of the choice of the initial distribution functions at t=0. The presented results are obtained with initially uncorrelated distribution functions.

In the pair approximation, Eq. (21) becomes in the steady state, with Dₖₗ=0,

$$- 2 S ( \text{?} ) g ( \text{?} ) + \frac{2 G}{T \text{?}} ( 1 - g ( \text{?} ) ) - \text{?} \qquad ( 38 )$$

$$\text{?} \text{indicates text missing or illegible when filed}$$

which leads to

$$g ( \text{?} ) = \frac{1}{1 + \frac{\text{?}}{\text{?}}} . \qquad ( 39 )$$

$$\text{?} \text{indicates text missing or illegible when filed}$$

Inserting this into Eq. (23) and putting dT/dt=0, we obtain:

$$- \text{?} T + G ( \frac{1}{\text{?}} - T ) - \text{?} \frac{GS ( \text{?} )}{G + T \text{?} S ( \text{?} )} = 0 \text{?} \qquad ( 40 )$$

$$\text{?} \text{indicates text missing or illegible when filed}$$

We can, again in very good approximation, replace the lattice sum by an integral:

$$( 41 )$$

$$\text{?} \frac{GS ( \text{?} )}{G + T \text{?} S ( \text{?} )} \approx \frac{G \text{?}}{\text{?}} \int dr {( \frac{\text{?}}{r} )}^{6} \frac{1}{G + T \text{?} {( \frac{\text{?}}{\text{?}} )}^{6}} = \frac{2}{3} \sqrt{\frac{G \text{?}}{T \text{?}}} {π}^{2} {( \frac{\text{?}}{\text{?}} )}^{3} ,$$

$$\text{?} \text{indicates text missing or illegible when filed}$$

so that Eq. (40) becomes

$$- \text{?} T + G ( \frac{1}{\text{?}} - T ) - \frac{2}{3} \text{?} \sqrt{G \text{?}} {( \frac{\text{?}}{\text{?}} )}^{3} = 0. \qquad ( 42 )$$

$$\text{?} \text{indicates text missing or illegible when filed}$$

This equation can easily be solved numerically to obtain T (G). The 2D equivalent of the lattice sum Eq. (41) is

$$\text{?} \frac{GS ( \text{?} )}{G + T \text{?} S ( \text{?} )} = \frac{2}{3 \sqrt{3}} ( \frac{{G}^{2} \text{?}}{{T}^{2} \text{?}} \text{?} {π}^{2} {( \frac{\text{?}}{\text{?}} )}^{2} , \qquad ( 43 )$$

$$\text{?} \text{indicates text missing or illegible when filed}$$

so that the 2D equivalent of Eq. (42) for the steady state PA triplet density becomes

$$- \text{?} T + G ( \frac{1}{\text{?}} - T ) - \frac{2}{3 \sqrt{3}} \text{?} {π}^{2} ( {G}^{2} \text{?} {( \frac{\text{?}}{\text{?}} )}^{2} = 0. \qquad ( 44 )$$

$$\text{?} \text{indicates text missing or illegible when filed}$$

In the superposition approximation, Eq. (22) becomes in the steady state, with Dₖₗ=0,

$$- 2 S ( \text{?} ) g ( \text{?} ) + \frac{2 G}{T \text{?}} ( 1 - g ( \text{?} ) ) - T \text{?} ( S ( \text{?} ) + S ( \text{?} ) ) g ( \text{?} ) g ( \text{?} ) g ( \text{?} ) + 2 T \text{?} g ( \text{?} ) \text{?} S ( \text{?} ) g ( \text{?} ) = 0 \text{?} \qquad ( 45 )$$

$$\text{?} \text{indicates text missing or illegible when filed}$$

In principle, this equation can be solved directly, in conjunction with Eq. (23) after putting dT/dt=0. Instead, we use the same practical approach as for the 2^{nd} and 3^{rd} order approximation and solve the corresponding time-dependent coupled differential equations until no further change in time is observed.

### D. Triplet Diffusion

In this subsection we include diffusion of triplet excitons. We will not again consider in this case the 2^{nd} and 3^{rd} order approximation, since we will see in the next section that for the case without diffusion these approximations turn out to be considerably less accurate than the pair and superposition approximation.

Since diffusion of triplets can only occur among guest sites, we encounter the problem of percolation, where fast exciton diffusion can occur along percolating pathways of guest sites that happen to be close together. This is a problem that we cannot fully address without sacrificing translational symmetry. Instead, we preserve translational symmetry and use an approximate approach, which is based on the idea that the fraction of sites available for diffusion is c_{g} and that the typical minimal distance over which triplet transfer can occur is therefore c_{g}^{-1/3} a instead of a. This approximation neglects the fact that there can be percolating pathways for triplet transfer with nearest-neighbor guest sites closer than this distance. Therefore, our approach will underestimate triplet diffusion. However, because of the long-range character of the Forster transfer, this underestimation is not severe. The other approximation we make is the replacement of the lattice description by a continuum description. In this continuum description, the discreteness of the underlying lattice is reflected by a minimal distance r₀ of the order of the lattice constant a over which TTA can occur, which is the same as in the case of no diffusion. We choose the value of r₀ such that Forster-type sums can be replaced by corresponding integrals:

$$\text{?} \frac{1}{\text{?}} = \text{?} dr \text{?} \frac{\text{?}}{\text{?}} = \frac{\text{?}}{3} ( \frac{\text{?}}{\text{?}} \text{?} = 8.402 \text{?} \qquad ( 46 )$$

$$\text{?} \text{indicates text missing or illegible when filed}$$

where we have performed the same lattice sum as in Eq. (27). From this we find r₀=0.7929a. Accordingly, a minimal distance [symbol] =c_{g}^{-1/3}r₀ is introduced over which triplet transfer can take place. With the above approximations Eq. (19) transforms into

$$\frac{dT}{dt} = - \text{?} + G ( \frac{1}{\text{?}} - T ) - \text{?} {T}^{2} \text{?} dr \text{?} S ( \text{?} ) g ( \text{?} ) . \qquad ( 47 )$$

$$\text{?} \text{indicates text missing or illegible when filed}$$

In the MF approximation we have g(r, t)=1 and we obtain exactly the same results as without diffusion, discussed in the previous subsection, both in the transient and steady state case and irrespective of the guest fraction c_{g}. The PA for the two-triplet correlation function Eq. (21) turns into:

$$\frac{\text{?} ( \text{?} )}{dt} = - 2 S ( \text{?} ) g ( \text{?} ) + \frac{2 G}{T \text{?}} ( 1 - g ( \text{?} ) ) - 2 g ( \text{?} ) \frac{\text{?}}{\text{?}} \text{?} D ( \text{?} ) + 2 \frac{\text{?}}{\text{?}} \text{?} D ( \text{?} ) g ( \vert \text{?} - r \vert , t ) , \qquad ( 48 )$$

$$\text{?} \text{indicates text missing or illegible when filed}$$

Where \δVᵣ and \\tilde{V}ᵣ indicate that a spherical volume with a radius r₀ and [symbol] , respectively, around r is excluded from the integrals over the infinite volume V. The last two terms have a prefactor c_{g} because the density of sites available for diffusion is reduced by that factor. The SA Eq. (22) becomes

$$\frac{dg \text{?}}{d \text{?}} = - 2 S ( \text{?} ) g ( \text{?} ) + \frac{2 G}{T \text{?}} ( 1 - g ( \text{?} ) ) + 2 Tg ( \text{?} ) \text{?} S ( \text{?} ) g ( \text{?} , t ) - Tg ( \text{?} , t ) \text{?} [ S ( \text{?} ) + S ( \vert \text{?} \vert ) ] g ( \text{?} , t ) g ( \vert \text{?} \vert t ) - 2 g ( \text{?} ) \frac{\text{?}}{\text{?}} \text{?} D ( \text{?} ) + 2 \frac{\text{?}}{\text{?}} \text{?} D ( \text{?} ) g ( \vert \text{?} \vert t ) \text{?} \qquad ( 49 )$$

$$\text{?} \text{indicates text missing or illegible when filed}$$

We obtain transient results from Eqs. (47) and (48) (PA) or (49) (SA) by putting G=0 and solving the resulting equations numerically, where the integrals are evaluated using a spectral method. For numerical reasons we set the pair correlation function g(r, t)=1 when r≥250 nm, which means that we neglect correlations for larger distances. We checked that this is a sufficiently large cutoff on the correlations. Steady state results are obtained by including the terms with G and solving the equations until no further change in time is observed, as we did for the case without diffusion.

### III. Results

### A. No Triplet Diffusion

In this subsection, we discuss the impact of triplet correlations on triplet-triplet annihilation (TTA) in time-resolved photoluminescence (TRPL) experiments and steady state conditions in the absence of triplet diffusion. In the simulation of TRPL experiments, the triplets are initially randomly positioned. In the absence of triplet diffusion, the value of the guest concentration is then irrelevant, because also the positions of the phosphorescent guest molecules are taken to be random. In steady state conditions, we assume that there is a constant generation rate of triplet excitons, randomly at guest molecules that are not yet occupied by another triplet exciton.

Plots 201-204 of FIG. 2 show the time-dependent triplet density T(t) in simulations of a TRPL experiment with an initial triplet density T₀=0.02 nm⁻³ for two different TTA Förster radii R_{F,TTA}=2.0 nm, shown in plots 201 and 202, respectively, and 3.0 nm, shown in plots 203 and 204, respectively. Each plot shows the transients for mean field (MF), 2nd order, 3rd order, Pair (PA), and superposition (SA) approximation, and KMC simulations. For a typical phosphorescent guest concentration of 10%, T₀=0.02 nm⁻³ corresponds to 20% initial occupation of the guest molecules by a triplet exciton. Time t is expressed in units of the triplet exciton lifetime T=1/kᵣ. The measured photoluminescence is proportional to T(t). We show transients up to t=2T, which is a typical time range in TRPL experiments. Plots 201 and 203 are semi-log and plots 202 and 204 are log-log plots of the same data and the insets in plots 202 and 204 are magnifications of the indicated regions. We compare the transients for the five approximations discussed in Subsec. IIB to those of kinetic Monte Carlo (KMC) simulations discussed in Subsec. IIA, which serve as a benchmark. The mean field (MF) and pair approximation (PA) transients are given by the analytical expressions Eqs. (28) and (33), respectively.

The effect of TTA is clearly seen in all transients from the non-exponential decay. After some time, triplets that are initially close to each other will have undergone TTA, creating correlations in the positions of the triplets as sketched in FIG. 1. For long times, the surviving triplets become isolated and TTA becomes unimportant, as observed by the exponential decay at long times, as most clearly visible in plots 201 and 203. We see that the MF transients, obtained from Eq. (28), start to strongly deviate from the KMC transients after t=10-27 for R_{F,TTA}=2.0 nm and t≈10⁻³_{T} for R_{F,TTA}=3.0 nm; see plots 202 and 204, respectively. The reason for the deviation is that correlations in the positions of the triplets are neglected in the MF approximation, which leads to an overestimation of TTA and a too strong initial decrease of T(t). The 2^{nd} and 3^{rd} order transients are increasingly accurate, but even the 3^{rd} order transients finally deviate from the KMC transients. On the other hand, the transients for the pair approximation and the superposition approximation (SA) follow the KMC transients quite accurately. The magnification in plot 204 shows that for R_{F,TTA}=3.0 nm and times approaching t=T a small deviation of the PA from the KMC transient becomes visible, while the SA transient still closely follows the KMC transient. The magnification in plot 202 shows that for R_{F,TTA}=2.0 nm also the PA transient keeps on following the KMC transient quite closely.

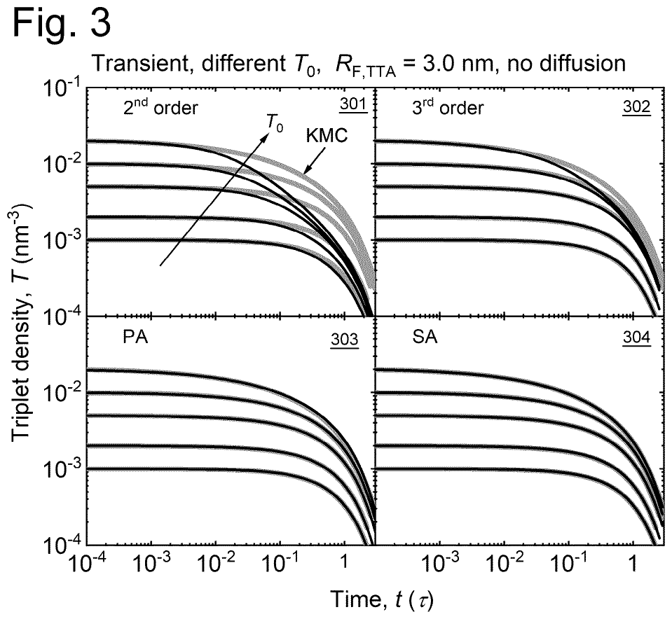

FIG. 3 shows transients for R_{F,TTA}=3.0 nm and different initial triplet exciton densities T₀ between 10⁻³ and 2×10⁻² nm⁻³. The transients are shown for initial triplet densities T0=10−3, 2×10−3, 5×10−3, 10−2, and 2×10−2 nm−3, in absence of triplet diffusion. Plots 301 and 302 of FIG. 3 show transients for the 2^{nd} and 3^{rd} order approximations, respectively, and plots 303 and 304 for the PA and SA, respectively, in comparison to the KMC transients. We see that the 2^{nd} and 3^{rd} order transients deviate less from the KMC transients at lower T0, which is related to the fact that at lower initial triplet densities correlation effects are less important. As expected, the 3^{rd} order transients are superior to the 2^{nd} order transients. The PA and SA transients follow the KMC transients very closely for all values of T₀.

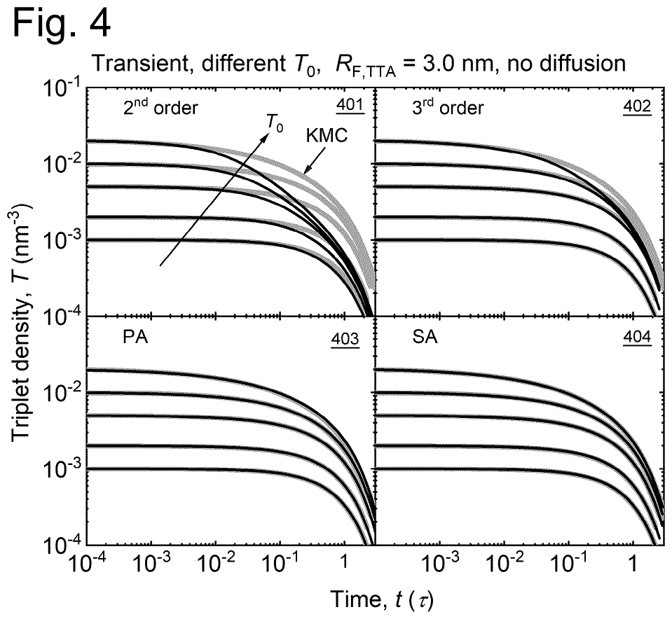

In plots 401-404 of FIG. 4 we show the triplet pair correlation function g(r, t) (Eq. (24)) at various distances r as a function of t for R_{F,TTA}=3.0 nm and a high initial triplet density of T0=2×10⁻² nm⁻³, for which the effects of TTA are relatively strong. We show results for the same approximations as in FIG. 3, in comparison to KMC results. The values of r correspond to the possible distances between points of the used cubic lattice with lattice constant a=1 nm. Initially, we have g(r, t=0)=1, which expresses the fact that the triplets are initially randomly positioned without any correlation. When time proceeds, a depletion zone with a radius of about RF (2kᵣt)^{1/6} (see Eq. (30)) develops around each triplet by the TTA process, where the probability of finding another triplet is reduced.

Finally, g(r, t) becomes zero for all r, which happens at a later time for larger r, indicating the presence of a growing fully developed depletion zone. The accuracy with which the transients of the various approximations in FIG. 3 follow the KMC transients is reflected in the accuracy with which the corresponding correlation functions follow the KMC correlation function. The PA correlation function in plot 403, given by Eq. (30), is a substantial improvement, yielding only small deviations from the KMC correlation function at long distances. The SA correlation function in plot 404 shows an excellent agreement.

We now turn to the steady state situation where triplets are generated randomly, at positions where there is no other triplet, with a rate G. This corresponds to an experiment under continuous illumination or to the situation where triplets are homogeneously generated by electron-hole recombination in an emission layer of an OLED. In the latter case, we neglect the interaction of the triplets with the electrons and holes.

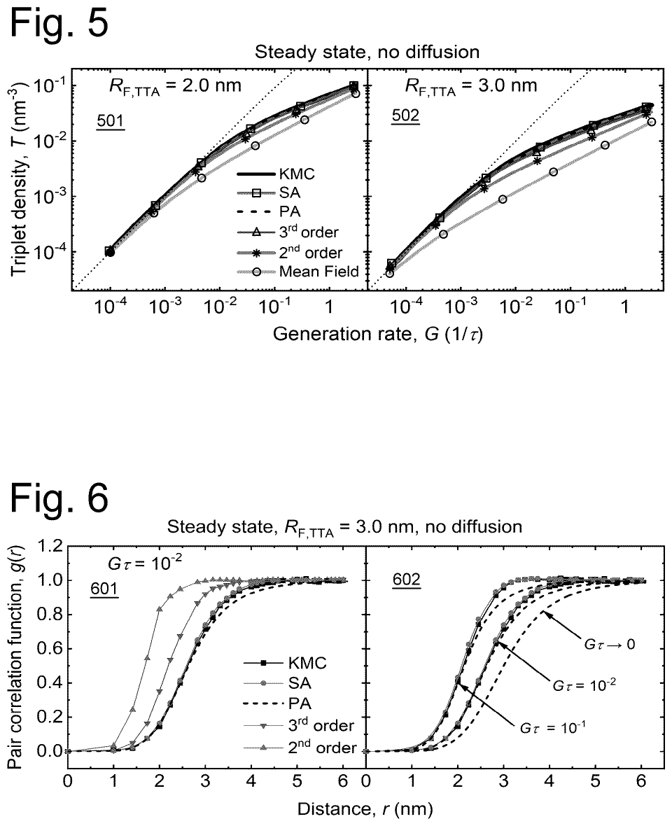

In plots 501 and 502 of FIG. 5 we show the steady state triplet density T as a function of the generation rate G for R_{F,TTA}=2.0 and 3.0 nm, respectively, for the five approximations of Subsection IIB, in comparison to KMC results. The MF result is given by the analytical expression Eq. (37). The PA result is obtained by solving the analytical expression Eq. (42). For a low generation rate, TTA is insignificant, so that all results approach the dotted line T=G_{T}a₋₃/(1+G_{T}) for low G. For high G all results approach each other, pointing at a decreased significance of correlations. Large differences between the various results are observed for intermediate values of the generation rate, where for realistic values G_{T}=10⁻² and R_{F,TTA}=3.0 the MF result underestimates the triplet density by almost an order of magnitude. Apart from the MF and 2^{nd} order approximations, the other approximations (3^{rd} order, PA, SA) yield results in very good agreement with the benchmark KMC results.

Plot 601 of FIG. 6 shows, for R_{F,TTA}=3.0 nm and G_{T}=10⁻², results for the pair correlation function g(r) for the four main approximations, 2^{nd} order, 3^{rd} order, PA, and SA, in comparison to KMC results. The underestimation of the size of the depletion zone by the 2^{nd} order approximation (green upward triangles) results in the underestimation of the triplet density by that approximation in FIG. 5. The 3^{rd} order approximation (orange downward triangles) partly corrects for this underestimation and hence yields better results for the triplet density in FIG. 5. The PA correlation function, given by Eq. (39), describes the KMC correlation function quite well, with only a small difference at large distances (cf. plot 403), while the SA yields an extremely good description. Plot 602 of FIG. 6 shows, in addition to results for G_{T}=10⁻² copied from plot 601, PA, SA, and KMC results for a larger exciton generation rate G_{T}=10⁻¹. The small deviation in g(r) at large distances of the PA has increased somewhat, while the SA still describes the KMC g(r) very well. The reduction of the depletion zone for a larger generation rate leads to a decreased role of correlations and hence to the convergence of all results for high Gr observed in FIG. 5. The reason is that for high Gr, the generation of excitons is so fast that there is insufficient time for the TTA process to generate correlations. We also added to plot 602 of FIG. 6 the result for the PA correlation function in the limit G_{T}→0, which is g(r)=1/[1+(R_{F,TTA}/r)⁶], as obtained from Eq. (39) after putting T=G_{T} a⁻³. It should be noted that in FIG. 6, apart from the PA case, the data points have discrete values of r that are connected by straight lines. It should be mentioned that these results are independent of T₀.

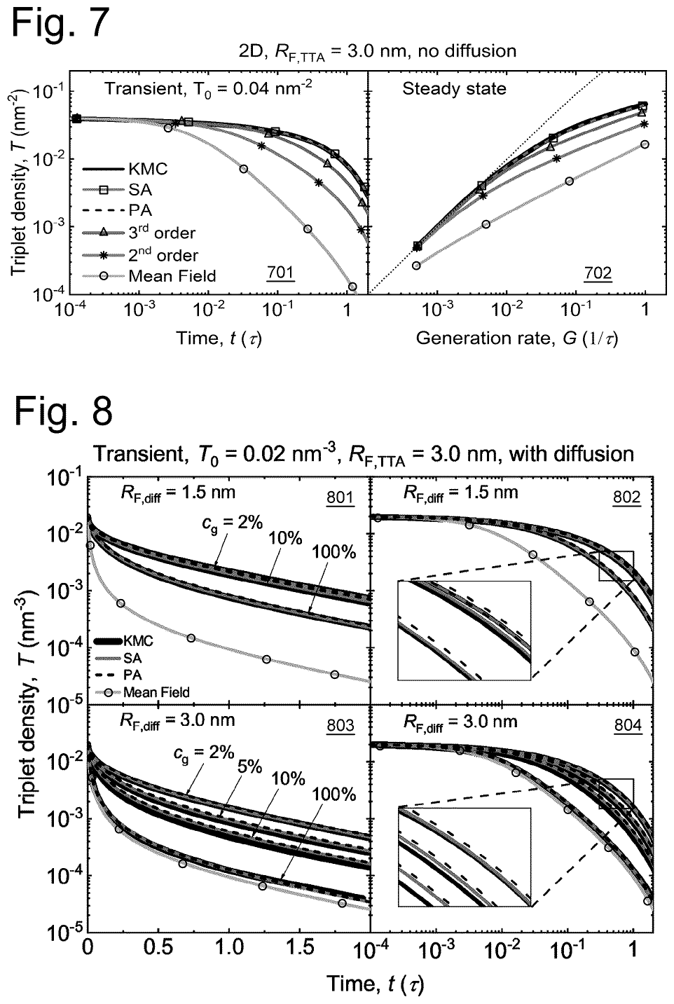

We have assumed thus far that excitons are generated homogeneously in three dimensions (3D), either by photoexcitation or by electron-hole recombination. In practice, there are situations where excitons are generated in a two-dimensional (2D) plane. This happens, for example, in thin emission layers or in situations where excitons are generated in the emission layer close to the interface with a charge transport layer. The latter can occur when one of the charge carriers in the emission layer has a much higher mobility than the other. For this reason, we also studied transient and steady-state triplet densities in a 2D square lattice. Analytical 2D results equivalent to the analytical 3D results can be found in Subsection IIC. Plot 701 of FIG. 7 shows transient triplet densities for R_{F,TTA}=3.0 nm and a 2D initial triplet density T₀=4×10⁻² nm⁻² for the five approximations discussed in Subsec. IIB in absence of triplet diffusion. For this initial triplet density, the average number of triplets within a disc of radius R_{F,TTA}=3.0 nm is 2.26, which is the same as the number of triplets within a sphere of this radius in the 3D case with T₀=2×10⁻² nm⁻³, which makes plot 701 comparable to plot 204. Plot 702 of FIG. 7 shows corresponding 2D steady-state results, which should be compared to plot 502. The dotted line shows the no-TTA limit T=G_{T}a⁻³/(1+G_{T}). The general conclusions that we have drawn about the 3D results also hold for the 2D results. A noticeable difference, however, is that the effects of correlations in 2D are stronger than in 3D, which is, e.g., seen from the larger deviation of the MF results from the KMC results in the comparison of plots 701 and 702 to plots 204 and 502, respectively. This is in line with the general conclusion in many-body theory that correlations become more important in lower dimensions.

### B. Triplet Diffusion

In this subsection, we consider the effects of triplet exciton diffusion on transient and steady-state triplet exciton densities in the presence of TTA. Because triplets can only diffuse among phosphorescent guests, it is now important to distinguish between guest sites, on which the triplets can reside, and host sites, which we assume to be inaccessible. The approximate approach followed to account for this is explained in Subsec. IID. This approach neglects percolation effects, where triplets can quickly diffuse along percolating pathways of guest molecules that happen to be close to each other. The approach therefore underestimates diffusion, but, as we will see, this underestimation is not severe.

FIG. 8 shows, for R_{F,TTA}=3.0 nm, an initial triplet density T₀=0.02 nm⁻³, and various guest concentrations c_{g}, the transient triplet density T(f) for R_{F,diff}=1.5 nm (plots 801 and 802) and R_{F,diff}=3.0 nm (plots 803 and 804). Plots 802 and 804 show the same as 801 and 802, respectively, but with a logarithmic time axis and insets in plots 802 and 804 are magnifications of the indicated regions. We give results for the mean field (MF) approximation, the pair approximation (PA), and the superposition approximation (SA), in comparison to KMC results. For intermediate guest concentrations, like c_{g}=5 and 10% in the case of R_{F,diff}=3.0 nm, the SA and PA transients are slightly above the KMC transients, which is related to the fact that our approach is not able to fully capture the effects of percolation and underestimates diffusion. For 100% guest concentration, percolation effects are absent, explaining why for this case the PA and SA transients very closely follow the KMC transients. For low guest concentration, like c_{g}=2%, the role of diffusion becomes small, explaining why for low guest concentrations the PA and SA transients also very closely follow the KMC transients. As seen in the insets in plots 802 and 804, the SA transients are more accurate than the PA transients. The displayed MF transients are identical for both values of R_{F,diff} and equal to the MF transient in plot 203 in the absence of diffusion. For R_{F,diff}=3.0 nm and 100% guest concentration, diffusion is so fast that correlations are almost completely washed out (see below). As a result, the MF transient is close to the PA, SA, and KMC transients, as seen in plots 803 and 804.

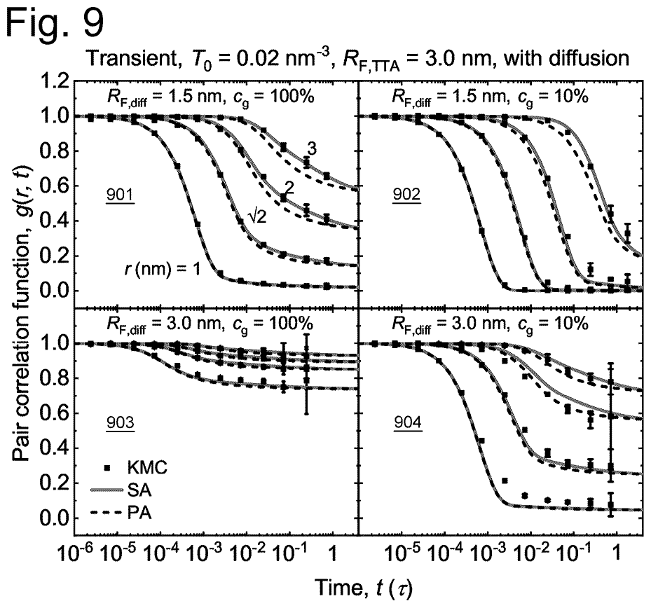

A comparison of the transient PA and SA pair correlation functions with the KMC pair correlation functions g(r, t) as a function of time for R_{F,TTA}=3.0 nm, T₀=0.02 nm⁻³, and four different values of r is shown in FIG. 9. Results are displayed for the four combinations R_{F,diff}=1.5 and 3.0 nm, and c_{g}=100% and 10%, wherein plot 901 shows results for R_{F,diff}=1.5 nm and c_{g}=100%, plot 902 shows results for R_{F,diff}=1.5 nm and c_{g}=10%, plot 903 shows results for R_{F,diff}=3.0 nm and c_{g}=100%, and plot 904 shows results for R_{F,diff}=3.0 nm and c_{g}=10%. A guest concentration of 10 mol % is typical for phosphorescent OLEDs. The case of 100% guest concentration could apply to single organic component fluorescent OLEDs, where the excitons are singlets instead of triplets. The results for the KMC correlation functions are given by the symbols, where standard error of the mean based on different boxes have been added. Because of the diffusion, there are much less triplets at later times in the simulation box than without diffusion. As a result, it is more difficult to obtain sufficient accuracy in the KMC correlation functions. We obtain a reasonable accuracy by performing an average of the correlation functions over finite time intervals. For this reason results are shown for much fewer times than for the case without diffusion, shown in FIG. 4. In all cases the PA and SA correlation functions are in quite good agreement with the KMC correlation functions. For 100% guest concentration (plots 901 and 903) the PA correlation function deviates somewhat from the KMC correlation function, while the SA correlation function is very accurate. For 10% guest concentration (plots 902 and 904) both the PA and SA correlation functions deviate somewhat from the KMC correlation functions, which should be attributed to the underestimation of diffusion by the neglect of percolation effects. The diffusion leads to incomplete depletion zones, with g(r, t) initially dropping, but finally leveling off at non-zero values, as observed for R_{F,diff}=1.5 nm and c_{g}=100% in plot 901, and R_{F,diff}=3.0 nm and c_{g}=10% in plot 904. For R_{F,diff}=3.0 nm and c_{g}=100% the diffusion is so strong that the depletion zone has almost disappeared; see plot 903. This is the reason why the MF transient in plot 803 is so close to the PA, SA, and KMC transients. On the other hand, for R_{F,diff}=1.5 nm and c_{g}=10% diffusion is weak, leading to an almost completely developed growing depletion zone, see plot 902, as observed without diffusion in FIG. 4.

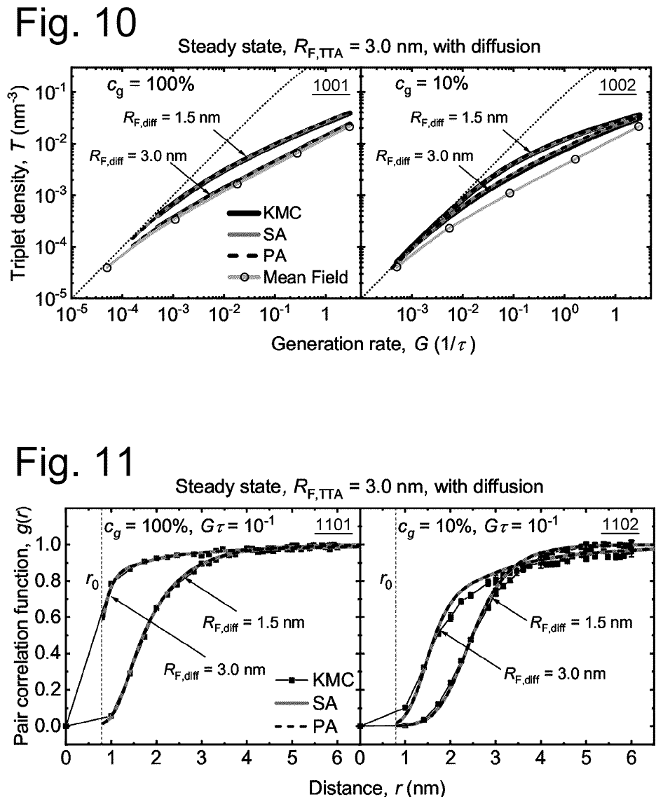

MF, PA, SA, and KMC steady-state results for the triplet exciton density T as a function of the generation rate G for R_{F,TTA}=3.0 nm, and R_{F,diff}=1.5 and 3.0 nm, are shown in plots 1001 and 1002 of FIG. 10 for c_{g}=100% and 10%, respectively. The dotted line shows the no-TTA limit T=G_{T}a⁻³/(1+G_{T}). Corresponding PA, SA, and KMC pair correlation functions are given in plots 1101 and 1102 of FIG. 11 for generation rates G_{T}=10⁻¹. For c_{g}=100%, both the PA and SA yield results in excellent agreement with the KMC results, both for the triplet densities as shown in plot 1001 and the correlation functions, shown in plot 1101. For c_{g}=100% and R_{F,diff}=3.0 nm, diffusion is so strong that even the MF result becomes reasonably accurate in plot 1001, like in plots 803 and 804 for the transient case. In plot 1101 we see that the correlations in this case are relatively unimportant. For c_{g}=10% and R_{F,diff}=1.5 nm the diffusion is weak and both the PA and SA results are very close to the KMC results and also very close to the results without diffusion in plot 502 (triplet densities) and plot 602 (correlation functions). For c_{g}=10% and R_{F,diff}=3.0 nm diffusion is important. The PA and SA results are then slightly above the KMC results in plot 1002, because of the underestimation of diffusion by the neglect of percolation effects. The diffusion is, however, not strong enough to completely wash out correlations, as seen plot 1102. As a result, the MF result for the triplet density in plot 1002 is not accurate. For c_{g}=10% and R_{F,diff}=1.5 nm diffusion is less important, leading to a more strongly deviating MF triplet density in plot 1002 and a larger depletion zone in plot 1102 as compared to R_{F,diff}=3.0 nm. Note that the KMC data points have discrete values of r and are connected by straight lines. For the PA and SA, g(r) is a continuous function defined for r>r₀=0.7929 nm.

### IV. Computational Efficiency

In this section, we compare the computational efficiency of pair-approximation (PA) and superposition-approximation (SA) calculations to that of KMC simulations. In the case of steady state calculations, the comparison is not straightforward, because of the different procedures used to reach the steady state. We therefore focus on a comparison for the transient calculations. We do not consider here the 2^{nd} and 3^{rd} order approximations, because these approximation are far less accurate than the PA and SA. The PA and SA calculations were done with Python codes that can be further optimized and possibly to some extent parallelized. The KMC simulations and SA and PA calculations were performed on comparable hardware (Intel Xeon Gold 6240 or comparable processors) with comparable numerical precision. We compare with CPU times for a single KMC run. For the KMC results presented in the previous section averages were performed over many KMC simulation boxes. Most of the transient results were obtained by taking an average over 50-100 KMC runs, apart from the results for T₀=10⁻³ and 2×10⁻³ nm⁻³ without diffusion, which required an average over 200 and 150 KMC runs, respectively.

For the case without triplet exciton diffusion we only considered the CPU time for SA calculations, since an analytic expression is available for the PA (Eq. (33)). We studied SA calculation and KMC simulation times for a TTA Forster radius R_{F,TTA}=3.0 nm as a function of the initial triplet density T₀ and for T₀=0.02 nm⁻³ as a function of the R_{F,TTA}. The CPU time for the SA calculations is independent of T₀, which is as expected, whereas it increases steeply with T₀ for the KMC simulations. For a typical initial triplet density T₀=0.02 nm⁻³ and Forster radius R_{F,TTA}=3.0 nm the KMC simulations take for a single run about a factor 4 longer than the SA calculations.

For the case with triplet exciton diffusion, where no analytical result is available for the PA, we studied the PA and SA calculation and KMC simulation times for R_{F,TTA}=3.0 nm and T₀=2×10⁻² nm⁻³ as a function of the guest concentration c_{g} with R_{F,diff}=1.5 and 3.0 nm. We find that the CPU time for the PA and SA is almost independent of c_{g}, where the CPU times for the PA calculations are about three orders of magnitude shorter than for the SA calculations. The CPU times are for both the PA and SA about the same for R_{F,diff}=1.5 and 3.0 nm. For the KMC simulations the CPU time increases approximately linearly with c_{g} and is significantly larger for R_{F,diff}=3.0 nm than for R_{F,diff}=1.5 nm. For a typical guest concentration c_{g}=10% the KMC simulations take for a single run about a factor 3 longer than the SA calculations for R_{F,diff}=1.5 nm, while this factor is an order of magnitude higher for R_{F,diff}=3.0 nm.

We remark that the KMC simulations can be straightforwardly parallelized by performing different runs on different processors, which makes the wall time in principle equal to that of a single run. But even then the speed-up by using PA or SA calculations is considerable and can be even further increased by optimizing and parallelizing the used codes.

### V. Summary

We applied master equation modeling to the description of Förster type triplet-triplet annihilation in organic emission layers consisting of a guest phosphorescent emitter embedded in a host, without and with inclusion of Forster type triplet exciton diffusion among the emitter molecules. We derived from the master equation for the time dependence of the probabilities for the different states of the system a hierarchical chain of equations that includes all correlations in the positions of the triplet excitons. Inspired by theoretical work on charge transport in disordered semiconductors given by the approach of Shumilin and Beltukov, we solved a chain of equations by neglecting covariances higher than 2^{nd} or 3^{rd} order. However, we found that these approximations yield insufficiently accurate results.

Instead, we showed that using a low-triplet density pair approximation (PA) or the superposition approximation (SA) yields accurate (PA) to very accurate (SA) results for relevant parameters, as benchmarked by kinetic Monte Carlo (KMC) simulations. This holds both in transient situations corresponding to time-resolved photoluminescence (TRPL) experiments as well as in steady state situations. The CPU time required for the SA or PA calculations can become, in some cases, orders of magnitude less than for the KMC simulations, which makes this type of master equation modeling an attractive alternative.

With only one type of excitation (triplet excitons) present, this work can be considered as a pilot study for taking into account the complicating effects of correlations between excitations in accurate yet fast calculations of the photophysics of OLEDs. With the inclusion of charges, application to triplet-polaron quenching (TPQ) should be possible. We further foresee application to electron-hole recombination and an application to charge transport that is more accurate than in the approach of Shumilin and Beltukov. With the inclusion of energetic and positional disorder, a complete accurate and fast alternative to KMC simulations of the photophysics of OLEDs may become available.

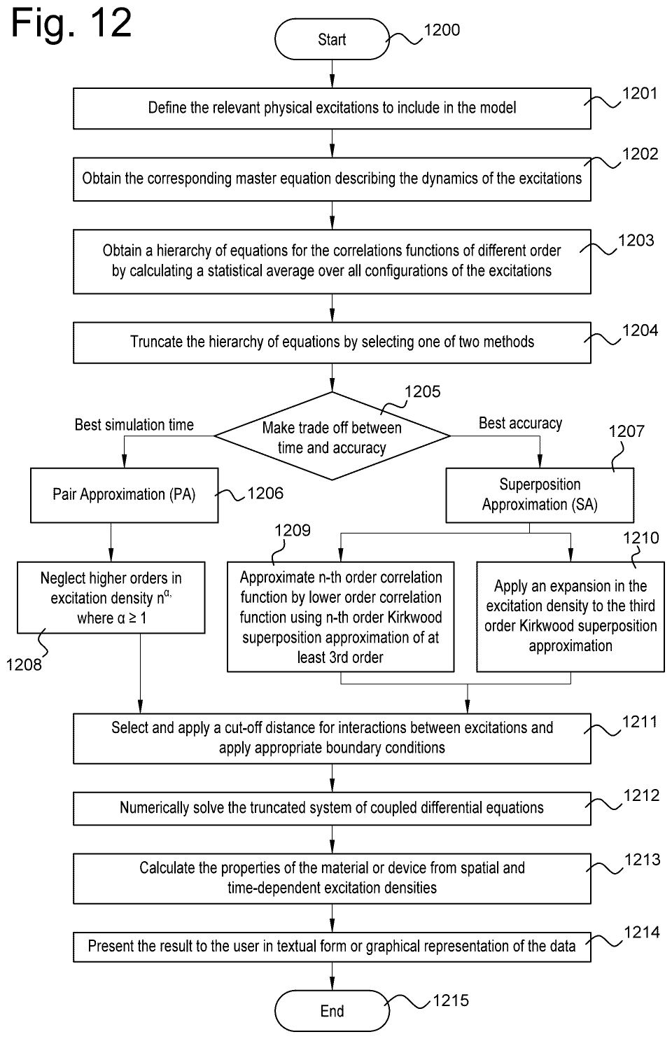

An overview of the simulation process is given in FIG. 12 which illustrates aspects of a method of determining a correlated evolution of a state relating to an object. The method starts at step 1200. An input to the method may include information indicative of a plurality of properties of the solid object, wherein the solid object comprises at least one material, and the plurality of properties comprise material properties of said at least one material. For example, the solid object may be laminated. Also, the different layers of the solid objects may be different compositions of materials. Further input to the method may be geometric information of the solid object, such as sizes and shape of the solid object and of its layers. Alternatively, the solid object may comprise regions of any shape with different composition and the geometric information may be indicative of those regions. Further input to the method may be information indicative of an external stimulus to which the solid object is exposed. An external stimulus may be, for example, light incident to the solid object or a voltage or current applied to the solid object. The light may be, for example, daylight or artificially generated light. The information relating to the external stimulus may comprise, for example, information relating to the type of stimulus, a strength of the stimulus, location of the stimulus, and/or direction of the stimulus.

The method may comprise the following steps.

In step 1201 the relevant physical excitations to include in the model are defined. This may include, for example, photoexcitation or electron-hole recombination, or triplet excitations.

In step 1202, a master equation is defined describing the time evolution of the probability that the system with its excitations is in a certain state in configuration space. This master equation depends primarily on the material composition, the geometry, and the type of excitations that are considered in step 1201. The master equation describes the solid object as a system of interacting excitations. The master equation may consider the transition rates between all the possible states of the system. The master equation may be determined, for example, using a method as disclosed in WO 2020/021008.

In step 1203, a set of hierarchical equations is determined that describes the dynamics of the system (i.e. the time evolution of the correlation functions of the excitations). For example, the set of hierarchical equations may be determined by applying an ensemble averaging or equivalent procedure. The set of hierarchical equations may be defined recursively.

To efficiently obtain an appropriate approximation of the outcome of the set of hierarchical equations, the hierarchical set of equations may be truncated. As shown in step 1205 there are at least two choices regarding the method to use for the truncation. For example the choice is between the pair approximation (PA) or the superposition approximation method (SA), as illustrated.

If the pair approximation is applied, in step 1206, the pair approximation method is chosen and applied. In this procedure, higher orders in excitation density may be neglected, as illustrated in step 1208. For example, a value a is set and higher orders with excitation density greater than n^{α} may be ignored. Herein, n (italic symbol n) denotes the occupation of a site within the solid object.

If the superposition approximation is applied, in step 1207, an expansion in the excitation density to the third order Kirkwood superposition approximation may be applied, as illustrated in step 1210. Alternatively, an n-th order correlation function is approximated by lower order correlation functions using an n-th order Kirkwood superposition approximation, wherein n (non-italic symbol n) is an integer value equal to or greater than 3, as illustrated in step 1209.

In step 1211, a cut-off distance may be applied for interactions between excitations. This may be performed by only considering exciton-exciton annihilation for sites separated by a distance smaller than a predetermined maximum distance.

In step 1212, the resulting truncated system of coupled differential equations may be numerically solved, for example by a computer simulation, taking into account the external stimulus. This step results in a correlated evolution of the state of the solid object.

Thereafter, as illustrated in step 1213, the resulting modelling method may be applied to the simulation of the correlation of the excitation dynamics in the system in such a manner that the system behavior and properties are predicted. For example, currents and voltages, heating properties, radiation (such as light) emitted by the solid object, may be calculated based on the determined evolution profile of the dynamics. In other words, the results may be used to compute properties, functionalities and performance of materials and devices, and assist in making decisions about device improvements, the design of new device structures or the synthesis of new molecules and materials.

In step 1214, results of the method are output. For example, as illustrated, the results may be displayed in text form or by means of a graphical representation. Alternatively, the results may be output as a signal that can be subjected to further processing. The user may benefit from the results with an accelerated R&D and manufacturing workflow and a reduction of costly and time-consuming trial-and-error experiments. For example, the user may manufacture a real version of at least one of the simulated solid objects.

One notable application of this method is for accelerating the modelling of organic (opto) electronic devices, for example organic light emitting diodes (OLEDs) or organic photovoltaics (OPVs) or photodetectors (OPDs), where correlation effects in the excitations are critical in determining the device behavior. Another application is for the modeling of charged species (e.g. ions) transport in batteries.

An aspect of the present invention provides a method to determine with sufficient accuracy the spatial correlations among interacting excitations within a physical system (the system), defined by a material or device, such that the results of this determination of the correlations may be applied to obtain an accurate description and ultimately an accurate prediction of the system behavior. The method comprises: The definition of a master equation describing the time evolution of the probability that the system with its excitations and interactions is in a certain state in configuration space. The application of an ensemble averaging or equivalent procedure to obtain a set of hierarchical equations that describes the dynamics of the system (i.e. the time evolution of the correlation functions of the excitations). The application of an approximation as closure for the hierarchical set of equations that define the model of the system. Obtaining the correlated evolution of the system state and the system properties by solving the equations of the model within a computer simulation for one or more types of excitations in the system. Said approximation may be, for example, the pair approximation (PA). Alternatively, said approximation may be the superposition approximation (SA).

The superposition approximation (SA) may be performed at the 3^{rd} order of accuracy. Alternatively, the superposition approximation (SA) may be performed at an order of accuracy higher than 3. Said material or device may be composed of multiple materials. Said device may be a semiconducting electronic device. Said device may be an organic electronic device. Said device may be among the following devices: light emitting diode, photovoltaic, photodetector, transistor, sensor, battery. Said excitations may be chosen among excitations involving excitons and/or charged particles. Additionally or alternatively, said interactions may be chosen among exciton-exciton annihilation and exciton-polaron quenching.

Some or all aspects of the invention may be suitable for being implemented in form of software, in particular a computer program product. The computer program product may comprise a computer program stored on a non-transitory computer-readable media. Also, the computer program may be represented by a signal, such as an optic signal or an electro-magnetic signal, carried by a transmission medium such as an optic fiber cable or the air. The computer program may partly or entirely have the form of source code, object code, or pseudo code, suitable for being executed by a computer system. For example, the code may be executable by one or more processors.

The examples and embodiments described herein serve to illustrate rather than limit the invention. The person skilled in the art will be able to design alternative embodiments without departing from the spirit and scope of the present disclosure, as defined by the appended claims and their equivalents. Reference signs placed in parentheses in the claims shall not be interpreted to limit the scope of the claims. Items described as separate entities in the claims or the description may be implemented as a single hardware or software item combining the features of the items described.

The invention claimed is:

1. A computer-implemented method of determining a correlated evolution of a state relating to a solid object subjected to a stimulus, the method comprising
obtaining information indicative of a composition and geometry of the solid object;
obtaining information indicative of an external stimulus in respect of the solid object;
determining a set of hierarchical equations representing dynamics of interacting excitations within the solid object, including spatial correlations among the plurality of excitations, based on the composition and geometry of the solid object, wherein the hierarchical equations are indicative of a time evolution of correlation functions of the excitations,
determining a closed set of hierarchical equations by closing the set of hierarchical equations according to an approximation; and
determining the correlated evolution of the state of the solid object based on the closed set of hierarchical equations and the external stimulus.

2. The method according to claim 1, wherein the solid object is an organic semiconductor device.

3. The method according to claim 1, further comprising predicting a physical behavior of the solid object based on the correlated evolution of the state of the solid object, wherein the physical behavior is associated with at least one of:
light emitted by the solid object,
light absorbed by the solid object,
energy stored within the solid object,
energy absorbed by the solid object, and
energy released by the solid object.

4. The method of claim 1, further comprising displaying a representation of the determined correlated evolution of the state of the solid object or a representation of the predicted physical behavior of the solid object.

5. The method according to claim 1, further comprising performing a measurement in respect of the solid object to generate at least part of the information.

6. A method of manufacturing a solid object with a particular physical behavior, comprising applying the computer-implemented method according to claim 1, and further comprising manufacturing the solid object in dependence on the determined correlated evolution of the state of the solid object.

7. The method according to claim 6, further comprising repeatedly applying the computer-implemented method according to claim 1 in respect of a plurality of different combinations of composition and geometry, and selecting the solid object to be manufactured based on the determined correlated evolution of the state of the solid object associated with each combination of composition and geometry.

8. A method of providing a stimulus to a solid object, comprising repeatedly applying the computer-implemented method according to claim 1 in respect of a plurality of different stimuli, and selecting a stimulus for the solid object, based on the determined correlated evolution of the state of the solid object associated with each of the plurality of different stimuli.

9. The method according to claim 1, wherein the determining the set of hierarchical equations comprises performing an averaging procedure, such as an ensemble averaging procedure, to a master equation that defines a time evolution of a probability that the solid object with its excitations is in a certain state in a configuration space.

10. The method according to claim 9, further comprising determining the master equation based on the plurality of properties of the solid object.

11. (canceled)

12. The method as claimed in claim 1, wherein said approximation comprises a pair approximation (PA).

13. The method as claimed in claim 1, wherein said approximation comprises a superposition approximation (SA).

14. The method as claimed in claim 13, wherein the superposition approximation (SA) is performed at a 3rd order of accuracy by taking into account covariances of up to 3 occupations and neglecting covariances beyond 3 occupations.

15. The method as claimed in claim 13, wherein the superposition approximation (SA) is performed at an order of accuracy higher than 3 by taking into account at least one covariance beyond 3 occupations.

16. The method as claimed in claim 1, wherein said solid object is composed of a plurality of different materials or said solid object comprises a semiconducting electronic device.

17. (canceled)

18. The method as claimed in claim 1, wherein said solid object forms at least part of an organic electronic device.

19. The method as claimed in claim 1, wherein said solid object comprises at least one of: a light emitting diode, a photovoltaic semiconductor, a photodetector, a transistor, a sensor, and at least part of a battery.

20. The method as claimed in claim 1, wherein the dynamics of interacting excitations represented by the set of hierarchical equations comprise interactions between at least two excitons, interactions between an exciton and a charged particle, or interactions between at least two charged particles.

21. The method as claimed in claim 1, wherein the dynamics of interacting excitations represented by the set of hierarchical equations comprise exciton-exciton annihilation or exciton-polaron quenching.

22. A system for determining a correlated evolution of a state relating to a solid object, the system comprising
at least one processor; and
a non-transitory computer readable media, comprising instructions that cause the at least one processor, when executing the instructions, to perform the steps of:
obtaining information indicative of a composition and geometry of the solid object;
obtaining information indicative of an external stimulus in respect of the solid object;
determining a set of hierarchical equations representing dynamics of interacting excitations within the solid object, including spatial correlations among the plurality of excitations, based on the composition and geometry of the solid object, wherein the hierarchical equations are indicative of a time evolution of correlation functions of the excitations,
determining a closed set of hierarchical equations by closing the set of hierarchical equations according to an approximation; and
determining the correlated evolution of the state of the solid object based on the closed set of hierarchical equations and the external stimulus.
